(19) United States
(12) Patent Application Publication
(10) Pub. No.: US 2009/0081226 A1
(43) Pub. Date: Mar. 26, 2009

(54) Soluble HLA-E Molecules And Their Use For Diagnosing And Treating Pathologies

(75) Inventors: Beatrice Charreau, Nantes (FR); Stephanie Coupel, Nentes (FR); Nadine Gervois, Nantes (FR); Laurent Derre, Lugrin (FR); Francine Jotereau, Carquefou (FR)

Correspondence Address: YOUNG & THOMPSON, 209 Madison Street, Suite 500, ALEXANDRIA, VA, 22314, US

(73) Assignee: INSERM (INSTITUT NATIONAL DE LA SANTE ET DE LA RECHERCHE MEDICLE), Paris (FR)

(21) Appl. No.: 11/792,251

(22) PCT Filed: Dec. 16, 2005
(86) PCT No.: PCT/EP2005/013565

§ 371 Date: Jul. 16, 2007

(30) Foreign Application Priority Data
Dec. 17, 2004 (EP) 04293023.0

(51) Int. Cl. A61K 39/395; C07K 14/00; C07K 1/00; C07K 16/18; C12Q 1/02; G01N 33/574; G01N 33/53; A61K 38/16

(52) U.S. Cl. 424/139.1; 530/350; 530/412; 530/387.9; 435/29; 435/7.23; 435/7.8; 514/12

(57) ABSTRACT

The present invention relates to a purified soluble HLA-E molecule, characterized in that:
it is a monomeric protein,
it presents a sequence identity of at least 80% with membrane-bound HLA-E (SEQ ID NO: 1),
it has a molecular weight from about 36 kDa to about 42 kDa,
it binds to the CD94/NKG2A receptor.

The classical MHC class I (Ia) molecules (HLA-A, HLA-B and HLA-C) are highly polymorphic and are ubiquitously expressed on most somatic cells. In contrast, non classical MHC class I (Ib) molecules (HLA-E, HLA-F and HLA-G) are broadly defined by a limited polymorphism and a restricted pattern of cellular expression.

Among class Ib molecules, HLA-E is characterized by a low polymorphism and a broad mRNA expression on different cell types (Lee et al. (1998) J Immunol 160, 4951-60). Cell surface expression of HLA-E requires the availability of β2-microglobulin (Ulbrecht et al. (1999) Eur J Immunol 29, 537-47) and of a set of highly conserved nonameric peptides derived from the leader sequence of various HLA class I molecules including HLA-A, -B, -C, and -G (Braud et al. (1997) Eur J Immunol 27, 1164-9; Ulbrecht et al. (1998) J Immunol 160, 4375-85). Efficient loading of HLA-E with class Ia leader sequence peptide requires the transporter associated with antigen processing (TAP) protein which translocates short peptides from the cytoplasm to the endoplasmic reticulum (Braud et al. (1998) Curr Biol 8, 1-10). HLA-E also associates with peptides which derive either from viruses, including cytomegalovirus (CMV), Epstein-Barr virus (EBV), and influenza virus, or from stress proteins (i.e. hsp60) (Ulbrecht et al. (1998) J Immunol 160, 4375-85; Tomasec et al. (2000) Science 287, 1031; Michaelsson et al. (2002) J Exp Med 196, 1403-14).

HLA-E tetramers binding to natural killer (NK) cells and a subset of T cells (αβ and γδ CD8 T cells), allowed the identification of CD94/NKG2A and CD94/NKG2C molecules as receptors for HLA-E (Braud et al. (1998) Nature 391, 795-9; Borrego et al. (1998) J Exp Med 187, 813-8; Lee et al. (1998) Proc Natl Acad Sci USA 95, 5199-204). The interaction of membrane-bound HLA-E with NK cells results in inhibition of NK cell-dependent lysis, mediated by the inhibitory CD94/NKG2A receptors (Braud et al. (1998) Nature 391, 795-9). CD94/NKG2A has also been implicated in down-regulation T-cell function in various pathological situations in humans such as melanoma (Speiser et al. (1999) J Exp Med 190, 775-82), ovarian carcinoma (Malmberg et al. (2002) J Clin Invest 110, 1515-23), arthritis (Dulphy et al. (2002) Int Immunol 14, 471-9) or astrocytoma (Perrin et al. (2002) Immunol Lett 81, 125-32). The function of the non-classical HLA-E molecules is not restricted to the modulation of NK cell responses as it also plays a role in the regulation of T cell function and represents a restriction element for the TCRαβ-mediated recognition (Pietra et al. (2001) Eur J Immunol 31, 3687-93; Li et al. (2001) J Immunol 167, 3800-8; Garcia et al. (2002) Eur J Immunol 32, 936-44; Heinzel et al. (2002) J Exp Med 196, 1473-81). HLA-E complexed with peptides can interact with αβTCRs expressed on CD8+T cells to trigger conventional CTL function Li et al. (2001) J Immunol 167, 3800-8; Lo et al. (1999) J Immunol 162, 5398-406; Speiser et al. (1998) Transplantation 66, 646-50; Leibson et al. (1998) Immunity 9, 289-94). Recent in vitro studies in human (Li et al. (2001) J Immunol 167, 3800-8) and the demonstration that Qa-1 (homologous to HLA-E in mice)-deficient mice (Hu et al. (2004) Nat Immunol 5, 516-23) have defects in immunoregulation mediated by CD8+T cells provide evidence of the involvement of HLA-E-restricted CD8 suppressor cells in controlling the adaptative immune response to both foreign and self antigens.

It is to be noted that the above-mentioned binding experiments between HLA-E and CD94/NKG2A have been mostly conducted with soluble HLA-E tetramers, mainly because the recombinant soluble HLA-E monomers used to date have been demonstrated to be inactive on CD94/NKG2A-expressing cells (Braud et al. (1998) Nature 391, 795-9). However, HLA-E tetramers are cumbersome to synthesize, since they result from the in vitro association of four bacterially-produced individual HLA-E molecules. Furthermore, their heavy molecular weight renders them unfit for in vivo use, and they lead to the inactivation of CD94/NKG2A-expressing cells.

Thus, one of the objects of the present invention is to provide a new soluble HLA-E molecule which is devoid of the defaults and inhibiting properties of the previously known soluble HLA-E molecules.

Another object of the present invention is to provide diagnostic and therapeutic methods using the new soluble HLA-E molecule.

The present invention relates to a purified soluble HLA-E molecule, characterized in that:

it is a monomeric protein,

it presents a sequence identity of at least 80% with membrane-bound HLA-E (SEQ ID NO: 1),

it has a molecular weight from about 36 kDa to about 42 kDa,

it binds to the CD94/NKG2A receptor.

HLA-E belongs to the HLA class I heavy chain paralogues. This class I molecule is a heterodimer consisting of a heavy chain and a light chain (β2-microglobulin). The heavy chain is anchored in the membrane. HLA-E binds a restricted subset of peptides derived from the leader peptides of other class I molecules. The heavy chain is approximately 45 kDa and its gene contains 8 exons. Exon 1 encodes the leader peptide, exons 2 and 3 encode the α₁ and α₂ domains, which both bind the peptide, exon 4 encodes the α₃ domain, exon 5 encodes the transmembrane region, and exons 6 and 7 encode the cytoplasmic tail.

As intended herein “soluble” notably relate to molecules which are not bound to lipidic membranes, in particular to cell membranes. In particular, “soluble HLA-E molecule” relates to a protein found as a cell-free protein released in the extracellular medium such as cell culture supernatant or biological fluids (i.e. serum, plasma, urine).

As intended herein “purified” relates to a protein which is substantially free of contaminants, such as other soluble HLA molecules such as classical Ia soluble HLA-A, B, C or non classical including MICA and MICB. The purification grade which can be obtained is of at least 95%.

Purity and specificity can be determined by methods well known to the man skilled in the art, such as SDS-PAGE and BB Coomassie staining and also by western blotting assays using HLA-E specific antibodies after protein transfer onto nitrocellulose membranes.

As intended herein the expression “monomeric protein” relates to a single protein or a protein complex which is not constituted of a plurality of identical protein chains covalently linked together; however the monomeric protein can be constituted of one or more different subunits. In particular, as intended herein, the monomeric soluble HLA-E molecule is constituted of a HLA-E heavy chain, or fragments thereof, optionally non-covalently associated to a β2-microglobulin subunit, or fragments thereof, this binary complex being itself optionally associated to a peptide; this whole association thus corresponds to a single MHC:peptide complex. It is, in particular different from HLA-E tetramers, which are constituted four specific MHC:peptide complexes bound to a single molecule of streptavidin.

As will be apparent to the man skilled in the art, the sequence identity percentage relates to the comparison of the sequence of the heavy chain of membrane-bound HLA-E with the corresponding chain of the soluble HLA-E molecule.

As intended herein the molecular weight of the proteins are determined by electrophoretically migrating sodium-dodecyl-sulfate-denatured protein samples on a polyacrylamide gel (SDS-PAGE), in particular as described in the following examples.

In vitro, the binding of the soluble HLA-E molecule to the CD94/NKG2A receptor can be assessed, for instance, by competition experiments involving the soluble HLA-E of the invention and known ligands of the CD94/NK2A receptor, such as antibodies or HLA-E tetramers.

In vivo, the binding of the soluble HLA-E molecule can be assessed according to the examples hereafter described.

The CD94/NKG2A receptor notably results from the association of CD94 (SEQ ID NO: 2) and NKG2A (SEQ ID NO: 3).

The present invention also relates to a process for obtaining a soluble HLA-E molecule, comprising a step of recovering soluble HLA-E molecules from a culture medium in which tumor cells, in particular melanoma cells, melanocytes, Natural Killer cells and/or endothelial cells have been grown.

The cells which produce soluble HLA-E can derive from immortalized cells lines or from primary cultures.

In a preferred embodiment, the invention relates to a process as defined above, wherein the culture medium contains at least one cytokine.

In a further preferred embodiment of the above defined process, the cytokine is selected from the list comprising IFNγ, IL1β and TNFα.

The present invention also relates to a soluble HLA-E molecule such as obtainable according to the above-defined process.

The present invention also relates to ligands of soluble HLA-E molecules as defined above which neither bind to membrane bound HLA-E molecules nor to bacterially produced soluble HLA-E molecules.

As intended herein, the above-defined ligands of soluble HLA-E molecules according to the invention are specific of the soluble HLA-E molecule according to the invention and do not bind to other known soluble HLA-E molecules, i.e. those recombinantly produced in bacteria, under a monomeric form, or resulting from the association from monomeric HLA-E molecules, which are recombinantly produced in bacteria, such as the previously known HLA-E tetramers for example.

According to a preferred embodiment, the above-defined ligands are selected from the list comprising antibodies or paratope-containing fragments thereof, and aptamers.

Preferably the antibodies are monoclonal antibodies.

As intended herein the expression “paratope-containing fragments” of antibodies notably relates to Fab, F(ab)′₂ or scFv fragments.

The expression “aptamers” relates to RNA molecules having specific binding capabilities vis-à-vis soluble HLA-E molecules.

The present invention also relates to an in vitro method for diagnosing cancers or inflammatory diseases in a patient, characterized in that HLA-E presence is detected in a biological sample, in particular a sample of solid tissues, such as skin, or liquid tissues, such as serum or plasma, taken from the patient.

In a particular embodiment of the above defined in vitro method for diagnosing cancers or inflammatory diseases in a patient, the biological sample is substantially depleted of cells which normally carry membrane-bound HLA-E, such as endothelial cells, B lymphocytes, T lymphocytes, macrophages, urothelial cells, secretory endometrial cells, or megakaryoblasts.

In a particular embodiment of the above defined in vitro method for diagnosing inflammatory diseases in a patient, the method is used for determining the disease activity or progression, particularly during inactive or active phase, relapse or remission of the disease.

In a preferred embodiment of the above defined in vitro method, HLA-E concentration is measured in a biological sample to test and compared to the HLA-E concentration in a normal corresponding biological sample, a higher HLA-E concentration in the biological sample to test as compared to the normal biological sample being indicative of a pathology.

By <<normal corresponding biological sample>> is meant a biological sample taken from a substantially healthy tissue, wherein the tissue is of the same histological type than the tissue from which the biological sample to test was taken.

HLA-E concentration can be measured according to methods well known to man skilled in the art, such as immunostaining or ELISA.

According to a preferred embodiment of the above defined in vitro method, the presence of soluble HLA-E is detected in a biological sample, in particular a sample selected from a sample of blood, serum, or plasma, taken from the patient

According to the present invention, soluble HLA-E is specifically present in biological samples of patients suffering from cancers or inflammatory diseases. It is essentially absent from samples of healthy patients.

In a preferred embodiment of the above defined in vitro method, soluble HLA-E concentration is measured in a biological sample to test and compared to the soluble HLA-E concentration in a normal corresponding biological sample, a higher soluble HLA-E concentration in the biological sample to test as compared to the normal biological sample being indicative of a pathology.

According to a further preferred embodiment of the above defined in vitro method, the presence of HLA-E is detected by contacting the sample taken from the patient with a HLA-E ligand.

According to another preferred embodiment of the above-defined in vitro method, the HLA-E ligand is selected from a list comprising anti-HLA-E antibodies or paratope-containing fragments thereof, anti-HLA-E aptamers, and CD94/NKG2A molecules or binding site-containing fragments thereof.

The expression “binding site-containing fragments” of CD94/NKG2A molecules relates to fragments of CD94/NKG2A which have retained its HLA-E binding capability.

According to a particularly preferred embodiment of the above-defined in vitro method, the cancers are melanomas, and the inflammatory diseases are vasculitides, in particular anti-neutrophil cytoplasmic antibody (ANCA)-associated systemic vasculitides.

In particular the ANCA-associated systemic vasculitides comprise Wegener's granulomatosis and microscopic polyangitiis. Anti-neutrophil cytoplasmic antibody (ANCA) associated systemic vasculitis (AASV) is a well-defined primary vasculitis subgroup, invariably associated with a systemic inflammatory response, which usually normalizes in remission. Thus AASV provides a useful clinical model to investigate the relation between clinical inflammation and endothelial dysfunction. Small vessel vasculitides, such as Wegener's granulomatosis (WG) and microscopic polyangiitis (MPA), are strongly associated with anti-neutrophil cytoplasmic antibodies (ANCA), which are either directed to myeloperoxidase (MPO) or proteinase 3 (PR3) (Cohen Tervaert J W et al, (1990) Kidney Int. 37: 799-806; Jennette J C and Falk R J (1997) N Engl J Med 337: 1512-1523; Velosa J A et al., (1993) Mayo Clinic Proc 68: 561-565). These diseases can occur in any organ system but the respiratory tract and the kidneys are most frequently involved. Untreated, WG results in death within weeks to months.

The present invention also relates to a kit for diagnosing cancers or inflammatory diseases in a patient, characterized in that it comprises:

at least one HLA-E ligand,

a mean for detecting the binding of the HLA-E ligand to a soluble HLA-E,

optionally a soluble HLA-E, in particular a soluble HLA-E as defined above, as a standard.

In a particular embodiment of the above defined kit, the HLA-E ligand is selected from a list comprising anti-HLA-E antibodies or paratope-containing fragments thereof, anti-HLA-E aptamers, and CD94/NKG2A molecules or binding site-containing fragments thereof.

In a particular embodiment of the above defined kit, the HLA-E ligand is an anti-HLA-E monoclonal antibody. Such an antibody can be used, for instance, for Western blotting experiments or for ELISA experiments.

The present invention also relates to a pharmaceutical composition, characterized in that it comprises at least one soluble HLA-E molecule as defined above in association with a pharmaceutically acceptable vehicle.

The present invention also relates to the use of at least one soluble HLA-E molecule as defined above for the manufacture of a medicament intended for the treatment of cancers, in particular melanomas.

In vivo, cancerous cell membranes present high quantities of membrane-bound HLA-E, which inactivates CD94/NKG2A-expressing cells, such as natural killer (NK) cells or cytotoxic T lymphocytes (CTL), which normally destroy cancerous cells. As demonstrated herein, the addition of soluble HLA-E according to the invention prevents CD94/NKG2A inactivation, which favours tumor destruction.

The present invention also relates to a pharmaceutical composition, characterized in that it comprises at least one HLA-E ligand, in particular at lest one soluble HLA-E ligand, more particularly at least one soluble HLA-E ligand as defined above, in association with a pharmaceutically acceptable vehicle.

In a preferred embodiment of the above-defined pharmaceutical composition, the HLA-E ligand is selected from a list comprising anti-HLA-E antibodies or paratope-containing fragments thereof, anti-HLA-E aptamers, and CD94/NKG2A molecules or binding site-containing fragments thereof.

The present invention also relates to the use of at least one HLA-E ligand for the manufacture of a medicament intended for the treatment of inflammatory diseases, in particular vasculitides, such as anti-neutrophil cytoplasmic antibody (ANCA)-associated systemic vasculitides.

Inflammatory diseases, such as vasculature inflammation (vasculitis), results, in particular, from an erroneous or exaggerated activation of NK cells or CTL. As demonstrated herein, erroneous or exaggerated activation of NK cells or CTL can be notably induced by soluble HLA-E molecules according to the invention produced by activated endothelial cells. Thus the use of ligands of soluble HLA-E molecules helps reduce the activation of NK cells or CTL.

In a preferred embodiment of the above-defined use, the HLA-E ligand is selected from a list comprising anti-HLA-E antibodies or paratope-containing fragments thereof, anti-HLA-E aptamers, and CD94/NKG2A molecules or binding site-containing fragments thereof.

In particular the HLA-E ligand is a soluble HLA-E ligand according to the invention.

The present invention also relates to the use of cytokines, in particular selected from the list comprising IFNγ, IL1β and TNFα, for producing a soluble HLA-E molecule from cells in vitro.

The present invention also relates to the use of a soluble HLA-E molecule as defined above or of a HLA-E ligand, for modulating the activity of CD94/NKG2A cells, in particular in vitro.

According to a preferred embodiment the invention relates to the above defined use of a soluble HLA-E molecule as defined above for activating CD94/NKG2A cells, in particular in vitro.

According to another preferred embodiment the invention relates to the above defined use of a HLA-E ligand, for inhibiting CD94/NKG2A cells, in particular in vitro.

According to a preferred embodiment of the above defined use, the HLA-E ligand is selected from a list comprising anti-HLA-E antibodies or paratope-containing fragments thereof, anti-HLA-E aptamers, and CD94/NKG2A molecules or binding site-containing fragments thereof.

### DESCRIPTION OF THE FIGURES

FIG. 1A, FIG. 1B, FIG. 1C, FIG. 1D, FIG. 1E
Comparative HLA-E cell surface expression in melanoma cell lines and melanocytes by flow cytometry

FIG. 1A: Flow cytometry profiles of HLA-E cell surface expression are shown on four melanoma cell lines (M28, M88, M204 and DAGI) untreated or following a 48 h incubation with IFNγ (500 U/ml). Cells were stained with isotype control (mouse IgG1) (light profiles), with anti-HLA-E mAb (MEM-E7) (black profiles) or with anti-HLA class I mAb (W6/32) (bold profiles).

FIG. 1B: The cell surface expression levels of HLA-E were evaluated on a panel of 22 untreated melanoma cells (dotted bars) and corresponding IFNγ-treated cells (black bars). The results represent semi quantitative analysis according to the ratio fluorescence intensity (RFI) obtained after flow cytometry.

FIG. 1C: Relative HLA-E (left) and total HLA class I (right) cell surface expression of 17 untreated melanoma cells (dotted bars) and IFN-γ-treated cells (black bars). The results represent semi quantitative analysis according to the ratio fluorescence intensity (RFI) obtained after flow cytometry. Asterisks represent the cell lines that produced soluble HLA-E upon IFN-γ treatment.

FIG. 1D: Flow cytometry profiles of HLA-E cell surface expression are shown on four short cultured melanocytes (00M33, 01M03, 01M10 and 01M11) untreated or following a 48 h incubation with IFNγ (500 U/ml). Cells were stained with isotype control (mouse IgG1) (light profiles), with anti-HLA-E (MEM-E7) (black profiles) or with anti-HLA class I mAb W6/32 (bold profiles).

FIG. 1E: Relative HLA-E (left) and total HLA class I (right) cell surface expression of 7 freshly isolated melanocytes (dotted bars) and IFN-γ-treated cells (black bars). The results represent semi quantitative analysis according to the ratio fluorescence intensity (RFI) obtained after flow cytometry. Asterisks represent the cell lines that produced soluble HLA-E upon IFN-γ treatment.

FIG. 2A, FIG. 2B, FIG. 2C, FIG. 2D, FIG. 2E, FIG. 2F, FIG. 2G FIG. 2H
In vivo HLA-E expression in melanoma lesions (immunoperoxidase staining of surgically removed tissues)
Comparative expression of HLA-E protein (MEM-E/02) was studied in normal skin (FIG. 2A), primary cutaneous melanomas (FIGS. 2C and 2D), melanoma invaded lymph node (FIGS. 2E and 2F), gastric metastatic melanoma (FIG. 2G) and hepatic metastatic melanoma (FIG. 2H). Staining of normal skin with Melan-A-specific mAb (A103) (FIG. 2B) was used as a melanocyte specific control. Magnification: ×400. Arrows indicate melanocytes (M) and endothelial cells (EC).

FIG. 3A, FIG. 3B, FIG. 3C, FIG. 3D, FIG. 3E, FIG. 3F, FIG. 3G, FIG. 3H, FIG. 3I, FIG. 3J
Immunoperoxidase staining showing HLA-E expression in human tissues
Comparative expression of HLA-E protein was studied in human first-trimester placenta tissue (FIG. 3A), kidneys (FIGS. 3B, 3C, 3D), spleen (FIG. 3E) and lymph node (FIG. 3F).
Comparative staining for HLA-G in placenta (trophoblast; FIG. 3G) and kidney (FIG. 3H). HLA-E expression in macrophages (M), megakaryocytes (MK) and sinus EC (sEC) in spleen (FIG. 3I) and in lymph node (FIG. 3J).
Magnification: ×400. Arrows indicate endothelial cells.

FIG. 4A, FIG. 4B, FIG. 4C, FIG. 4D, FIG. 4E, FIG. 4F
Cellular localization of HLA-E in human vascular endothelial cells—Immunofluorescence microscopy analysis of cultured vascular Ecs
Confocal microscope images showing comparative cell surface staining for HLA class Ia (HLA-A, -B, -C) (FIG. 4A) and HLA-E (FIG. 4B) on non permeabilized vascular endothelial cells. Nuclei were stained with To-pro-3. The colocalization of HLA-E (left panel), rhodamine-B hexyl ester (for ER staining) or anti-golgin-97 (for Golgi staining), both middle panel, was assessed on non-stimulated (FIGS. 4C and 4E) or IFNγ-activated (FIG. 4D and FIG. 4F) permeabilized ECs. Merged images are shown on the right panel. Magnification: ×63.

FIG. 5A, FIG. 5B, FIG. 5C, FIG. 5D, FIG. 5E, FIG. 5F, FIG. 5G, FIG. 5H, FIG. 5I
Up-regulation of cellular HLA-E and production of soluble HLA-E by cytokine-activated endothelial cells

FIG. 5A: Flow cytometry analysis of cell surface and intracellular HLA-E expression on HAEC at rest (medium) or after a 48 h-treatment with cytokines (TNFα, IFNγ, TNFα & IFNγ and IL-1β), by comparison with cell surface HLA-A2 expression. Cells were stained with anti-HLA-E (black profiles) or an isotype-matched control antibody (light profiles). Lower panel shows intracellular staining for HLA-E obtained after cell permeabilization. Mean of fluorescence intensity are indicated.

FIG. 5B: ECs were incubated with cytokines for 48 h, as above. Supernatants were collected and soluble HLA-E was then detected by western blotting in normal (1×) or concentrated (10×) supernatants (10 μl/sample). Results are representative of three independent experiments.

FIG. 5C: EC cultured for 18 h in the absence (medium) or in the presence of IFNγ were pre-incubated with cyclohexamide (CHX) for 1 h, or incubated with brefeldin A (BrfA) or metalloprotease inhibitor for the last 6 h of culture. Flow cytometry analysis of HLA-E expression was performed following immunostaining with MEM/E-7 mAbs (solid histograms) or an isotype-matched control antibody (histograms in dotted line). Mean of fluorescence intensity are indicated above.

FIG. 5D: Culture supernatants were collected, concentrated (10×) and analyzed as in FIG. 5B.

FIG. 5E: ECs were incubated with cytokines for 48 h or cultured for 18 h in the absence (medium) or in the presence of IFNγ, after a pre-incubation with cyclohexamide (CHX) for 1 h or with an incubation with brefeldin A (BrfA) for the last 6 h of culture. Supernatants were collected and sHLA-E was then detected by western blotting in normal (1×) or concentrated (10×) supernatants (20 μl/sample). Results are representative of three independent experiments.

FIG. 5F: Quantification and comparative analysis of mRNA steady state levels for HLA-A, HLA-B and HLA-E was assessed in cultured ECs by competitive RT-PCR. Values are mean±SD (n=3). *P<0.01 versus HLA-B.

FIG. 5G: Regulation of HLA-E mRNA in response to TNFα or IFNγ was assessed by semi quantitative RT-PCR. PCR amplifications for β-actin were used as control. RNA 18S and 28S are shown below.

FIG. 5H: HLA-E protein expression in untreated and IFNγ-activated for 48 h (400 U/ml) HUVEC and HAEC. Immunoblots were reprobed with anti-GAPDH mAb to compare protein loading within samples. A representative immunoblot is shown.

FIG. 5I: Flow cytometry profiles of HLA-E cell surface expression are shown on HUVEC and HAEC, either untreated or activated with IFNγ for 48 h (400 U/ml). Cells were stained with anti-HLAE (black profiles) or with an isotype-matched control antibody (white profiles). Mean of fluorescence intensity are indicated.

FIG. 6A, FIG. 6B
Up-regulation of cellular HLA-E by cytokine-activated endothelial cells isolated from various donors (FIG. 6A) and time-course production of sHLA-E by cytokine-activated endothelial cells (FIG. 6B)

FIG. 6A: Flow cytometry analysis of HLA-E expression on HUVEC and HAEC from donors (#8186, #11202, #9054, #14756) at rest (medium) or after a 48 h-treatment with cytokines (TNFα, IFNγ, IL1α). Cells were stained with an anti-HLA-E (histograms) or an isotype-matched control antibody (histograms in dotted line). Mean of fluorescence intensity are indicated.

FIG. 6B: Western blot analysis of sHLA-E production by HAEC after a time course treatment with TNFα and IFNγ (from 0 to 72 hrs).

FIG. 7A, FIG. 7B
Protection from CTL lysis of IFNγ-treated melanoma cell lines dependent of CD94/NKG2A/HLA-E interaction

FIG. 7A: The effect of IFNγ on CTL recognition of human melanoma cell lines was evaluated in a 4-hour ⁵¹Cr-release assay. T cell clones recognizing Melan-A/MART-1 antigen (Mel2.46 and M77.84) or NA17-A antigen (CDM39.91H and M17.221) were used as effectors. T cell clones represented on lower panel express the CD94/NKG2A receptor in contrast to the T cell clones represented on upper panel. Untreated melanoma cell line M204 (white) or treated (black) for 48 h with IFNγ (500 U/ml) was used as targets.

FIG. 7B: The same experiment was performed for two CD94/NKG2-A⁺ NA17-A specific T cell clones (CDM39.91A and H2) in the presence of the blocking anti-CD94 mAb (Y9) or of an irrelevant isotype-matched control Ab.

FIG. 8A, FIG. 8B, FIG. 8C, FIG. 8D, FIG. 8E
Detection of soluble HLA-E (sHLA-E) by Western blot analysis in culture supernatants of melanoma cell lines, melanocytes and serum from melanoma patient

FIG. 8A: The 48-h culture supernatants (lower panel) and the lysates (upper panel) of three melanoma cell lines pre-treated (+) or not (−) with IFNγ were subjected to SDS-PAGE, transferred to nitrocellulose membrane, and probed with mAb MEM-E2.

FIG. 8B: Kinetics analysis of the release of soluble HLA-E from melanoma cells. M200 cell line was cultured with 500 U/ml of IFNγ and the culture supernatants were collected at 12, 24, 48 and 72 h. HLA-E and GAPDH expression in the corresponding cell lysates is shown below.

FIG. 8C: Mechanism for the generation of the soluble HLA-E. M200 cells were stimulated by IFNγ for 24 h and cultured for a further 4 h with chloroquine, leupeptin, PMSF, EDTA or Galardin. Culture supernatants (upper panel) were harvested, subjected to SDS-PAGE, transferred to nitrocellulose membrane, and probed with mAb MEM-E2. Blot quantification (lower panel) was performed by densitometry analysis and expressed as arbitrary units (A.U.).

FIG. 8D: The 48-h culture supernatants of melanoma cell lines and short cultured melanocytes pre-treated or not with IFN-γ were subjected to SDS-PAGE, transferred to nitrocellulose membrane, and probed with mAb MEM-E/02.

FIG. 8E: A representative example of detection of sHLA-E by Western blot analysis in serum samples from melanoma patients. Sera from patients with melanoma (line 1: serum slightly positive; line 2: positive serum and line 3: negative serum) were subjected to SDS-PAGE, transferred to nitrocellulose membrane, and probed with mAb MEM-E2. An HLA-E-positive culture supernatant was used as positive control (Co+).

FIG. 9A, FIG. 9B, FIG. 9C
Activated NK cells produce soluble HLA-E (sHLA-E)

FIG. 9A: Fresh PBMC and purified NK subset were cultured for 48 h with Con A, IL-2 and anti-CD28 mAb or IFNγ. At the end of treatment, cells and culture supernatants were collected for flow cytometry and western blot analyses. Membrane-bound and soluble HLA-E (sHLA-E) were then detected by western in lysates (15 μg/sample) and supernatants (10 μl/sample), respectively. Immunoblots were reprobed with anti-GAPDH mAb to compare protein loading within samples.

FIG. 9B: For flow cytometry analysis of cell surface HLA-E expression, untreated PBMC cells were double stained with FITC-labeled anti-CD3, -CD4, -CD8, -CD14, or PE-labeled anti-CD19 mAbs and anti-HLA-E (MEM/E8) mAbs, revealed using a PE- or FITC-labeled anti-mouse secondary Ab. Results are expressed as dot plots after subset selection according to cytometric side scatter and forward scatter parameters. Results are representative of three independent experiments.

FIG. 9C: Cell surface expression of HLA-E was analyzed by flow cytometry on monocytoid (U937), T (Jurkat), B (Raji) and NK (NKL) cell lines. HLA-E staining (black profiles) was compared to labeling obtained using an isotype-matched irrelevant mAb (white profiles). The soluble HLA-E was detected by western blotting of culture supernatants from cell lines treated for 24 h with or without 150 U/ml of rIL-2.

FIG. 10A, FIG. 10B, FIG. 10C
Increase of lytic activity against HLA-E-expressing melanoma cells by soluble HLA-E (sHLA-E)

FIG. 10A: Incubation of CD94/NKG2-A⁺ T cell clone (H2) with sHLA-E-positive supernatant restores high killing of IFNγ-treated DAGI cells. As control, untreated or IFNγ-treated DAGI was killed at a similar level by a CD94/NKG2-A⁻ T cell clone (M17.221), independently of the presence or not of sHLA-E-positive supernatant.

FIG. 10B: Soluble HLA-E can enhance killing of M88 cell line by NK cells, a γδ T cell clone (C4.112) and more weakly by an αβ T cell clone (H2).

FIG. 10C: Increase of NK activity by sHLA-E can be reversed by addition of anti-HLA-E mAb. NK cells pre-incubated with sHLA-E-positive supernantant and anti-HLA-E mAb (MEM-E6) or IgG1 isotype were used as effector cells against untreated or IFNγ-treated M88 cells.

FIG. 11
Detection of soluble HLA-E (sHLA-E) in the sera of patients with vasculitis
Sera (10 μl /sample) from patients #1 to #9 were analyzed by western blot on a 12% SDS-PAGE. Immunoblots were incubated with an anti-HLA-E mAb (MEM/E2) and revealed with an anti-mouse-HRP.

FIG. 12A, FIG. 12B, FIG. 12C, FIG. 12D
Protective effect of cell surface and soluble HLA-E molecules against CD94/NKG2A-dependent NK cell cytotoxicity
In FIGS. 12A and 12C, purified NK were pre-incubated with culture medium (white column), irrelevant Ab (mouse IgG1) (10 μg/ml) (grey column) or anti-HLA-E mAb (10 μg/ml) (black column) for 20 min at RT.

FIG. 12A: Cytotoxicity assays were performed using target cells with no HLA-E expression at the cell surface, including the class I-deficient lymphoblastoid cell lines (C1R and K562) and primary cultures of SMC.

FIG. 12C: Cytotoxicity assays were performed using-ECs as target cells with a regulated HLA-E expression at the cell surface (untreated or activated with 100 U/ml IFNγ for 48 h). Target cells were labeled with 51Cr before incubation with NK cells for 4 h at 37° C. Results, expressed as mean of specific lysis±SD, are representative of three independent experiments. *p<0.01 versus untreated ECs, **p<0.01 versus cells incubated with medium or irrelevant Ab.

FIGS. 12B and 12D: Soluble HLA-E provides protection toward NK cell cytotoxicity to cells with no or low HLA-E expression at the membrane. Resting ECs (FIG. 12B) and SMC (FIG. 12D) were pre-incubated with culture medium (white circle) or conditioned medium from IFNγ-treated HAECs (black circle) for 20 min at RT, before incubation with freshly purified NK cells at various E:T ratios. Results, expressed as mean of specific lysis±SD, are representative of at least three independent experiments.

### EXAMPLES

### Antibodies

The following antibodies were used in the examples below:

Antibodies specific for CD94 (HP-3B1) and CD94/NKG2A (Z199) were purchased from Immunotech (Marseille, France). The CD94-specific antibody Y9 was kindly provided by A. Moretta (Genova, Italy). All flow cytometry stainings for CD94 were done with the mAb HP-3B1, while Y9 was used in the cytotoxic assays, as well as irrelevant mouse IgM Ab purchased from MedacGmbH (Hamburg, Germany).

For HLA-E staining we used antibodies MEM-E/2 for immunohistology and western blotting, MEM-E/6 for blocking experiments and MEM-E/7 or MEM-E/8 for flow cytometry, which were provided by V. Horejsi (Prague, Czech Republic) (Menier et al. (2003) Hum Immunol 64, 315-26). MEM-E/02 mAbs bind the denatured HLA-E protein whereas -E/06 (IgG1), -E/07 (IgG1) and -E/08 (IgG1) mAbs bind native cell surface HLA-E molecules. MEM-G/01 recognizes (similar to the 4H84 mAb) the denatured HLA-G heavy chain of all isoforms.

Anti-HLA class I antibodies (W6/32) were purchased from Immunotech (Marseillle, France).

### Example 1

### HLA-E Cell Surface Expression in Melanoma-Derived Cell Lines

The frequency of HLA-E cell surface expression in melanoma-derived cell lines was determined by flow cytometry.

The melanoma cell lines <<M>> used were mainly established from metastatic tumor fragments as previously described (Gervois et al. (1990) Eur J Immunol 20, 825-31). Other melanoma cell lines were obtained from different laboratories (IPC277/5, C. Aubert (Unite INSERM U119, Marseille, France); DAGI, J. F. Dore (Lyon, France); FM25 and FM29, J. Zeuthen (Copenhagen, Denmark); G-mel, A; Houghton (New York, USA), MW75, D. Schadendorf, Mannheim, Germany and Mel17, S. Perez (Athens, Greece). Normal melanocytes (00M10, 00M33, 011M03, 01M10, 01M11, 01M20 and 97M10) were obtained from M. Regnier (Clichy, France). All cell lines were cultured in complete medium (RPMI 1640 supplemented with 10% fetal calf serum (FCS)).

For flow cytometry, 10⁵ cells (melanoma cell lines or freshly isolated melanocytes) were stained with the anti-HLAE (MEM-E/07) mAb, anti-HLA class I (W6/32) mAb or the isotype control mAb for 30 min at 4° C. After two washes, cells were incubated for 30 min with the secondary PE-labeled antibody. Labeling was analyzed on a FACScan flow cytometer using Cellquest software (Becton Dickinson, Grenoble, France). 10 000 cells were gated with FSC/SSC parameters and analyzed. Ratio Fluorescence Intensity (RFI) has been calculated for HLA-E expression as follows: mean fluorescence intensity obtained with the test/mean fluorescence intensity obtained with the negative control.

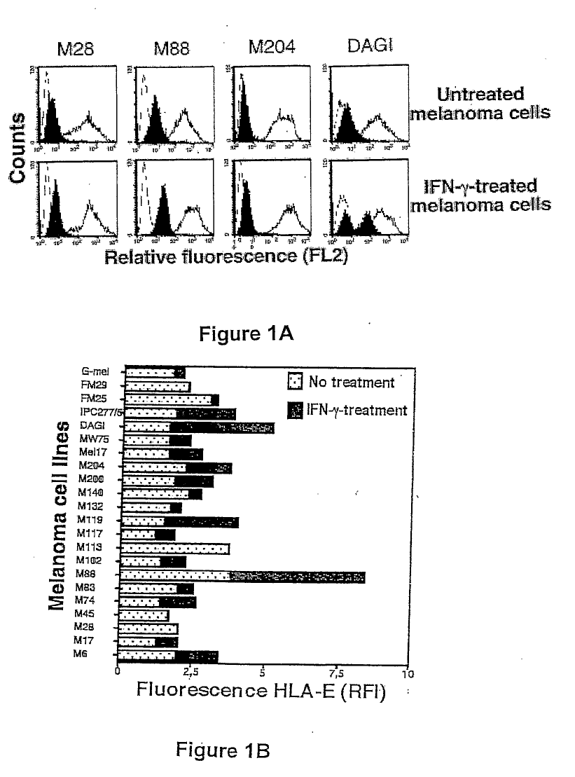

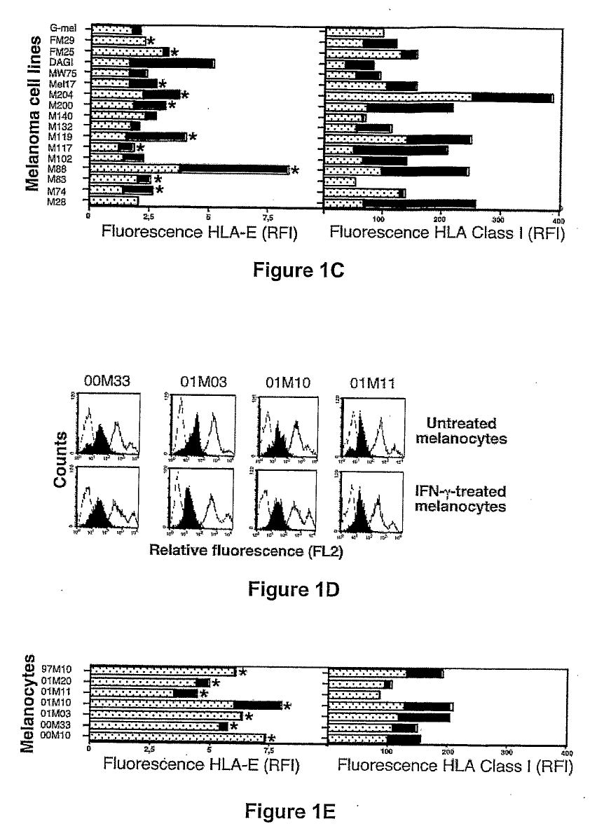

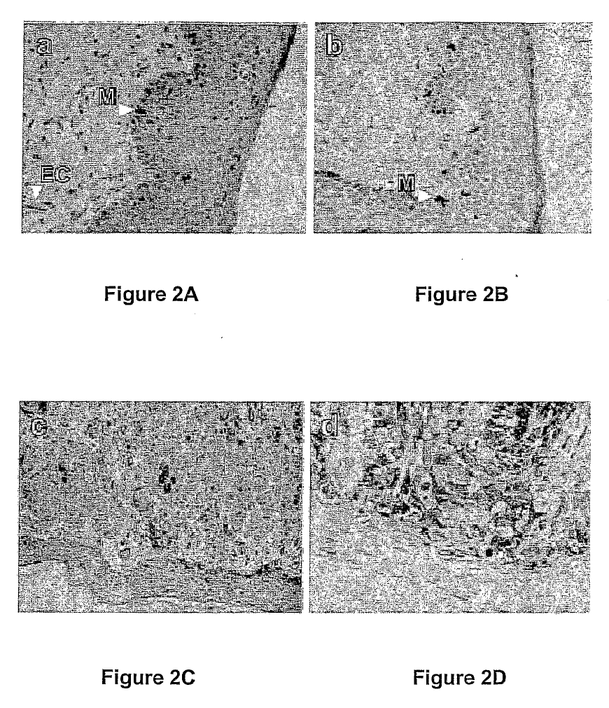

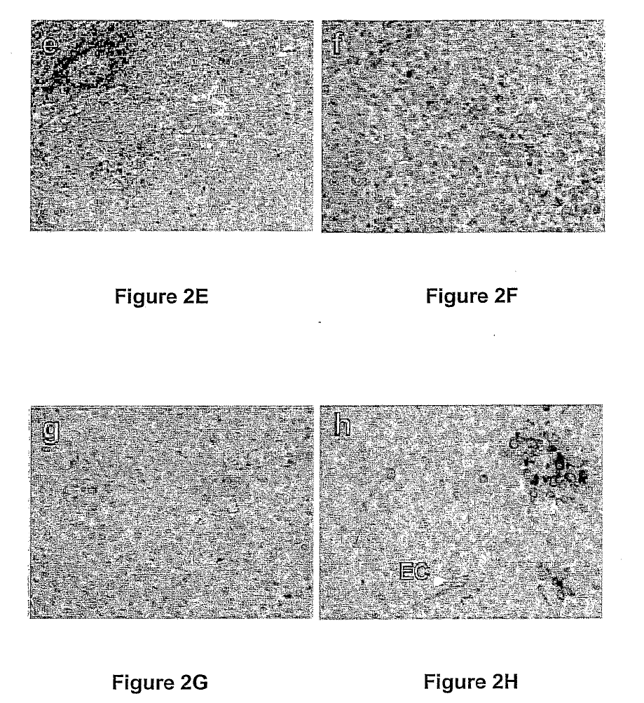

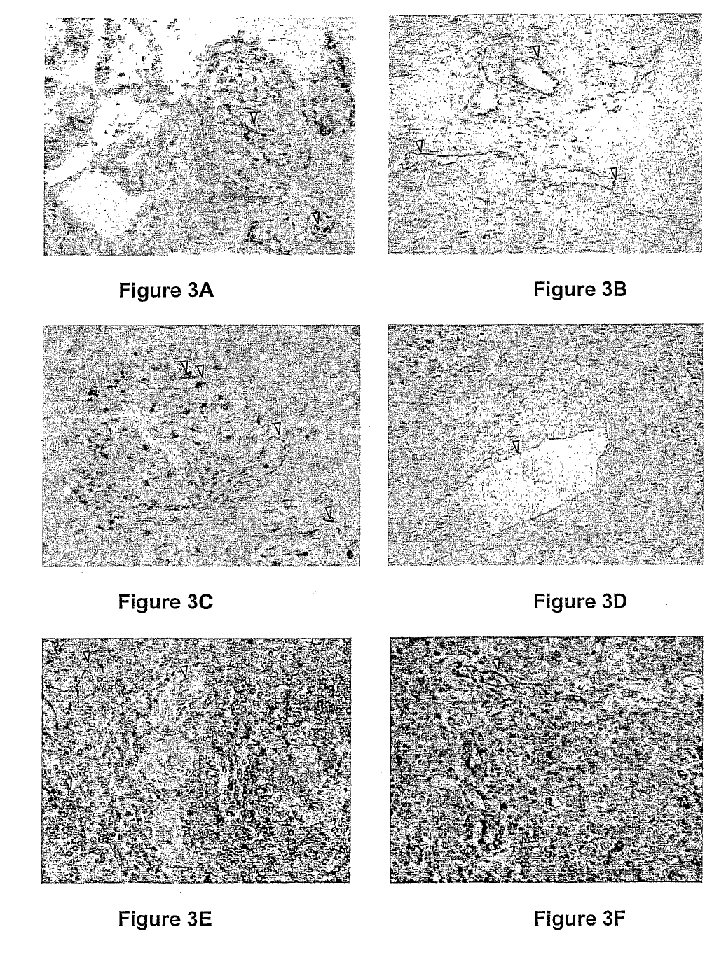

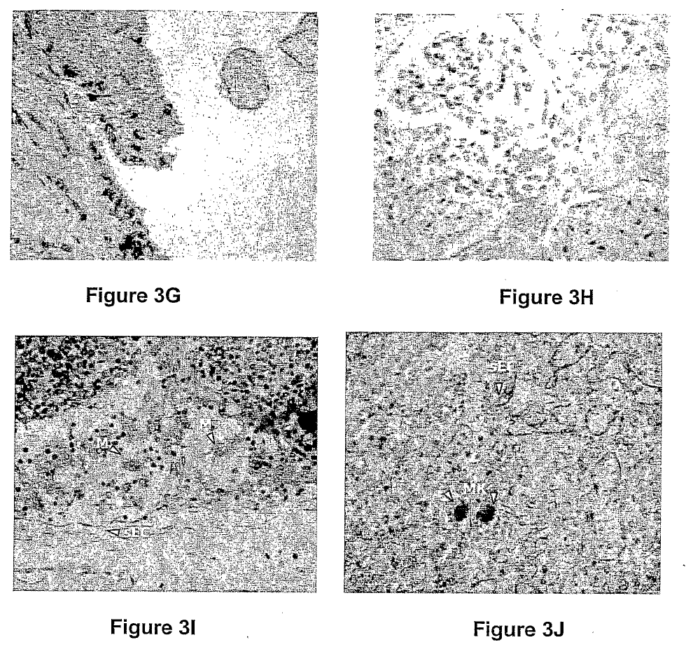

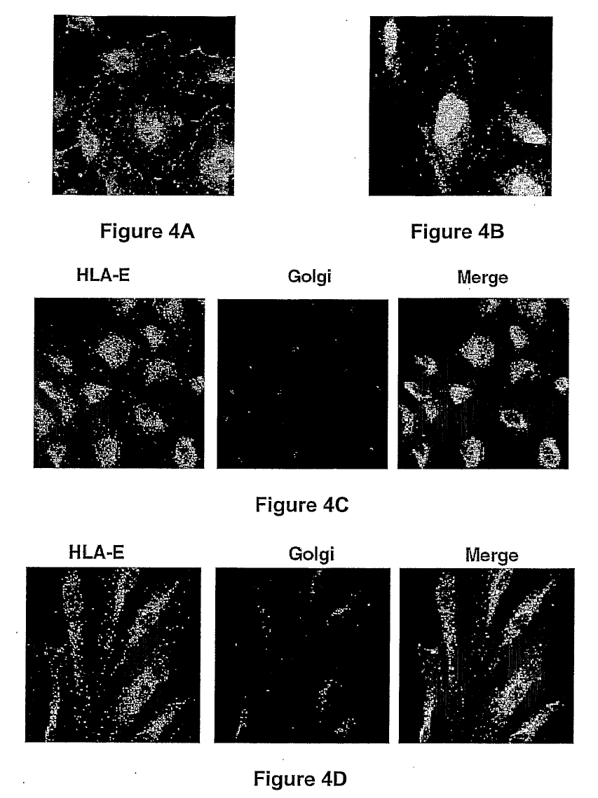

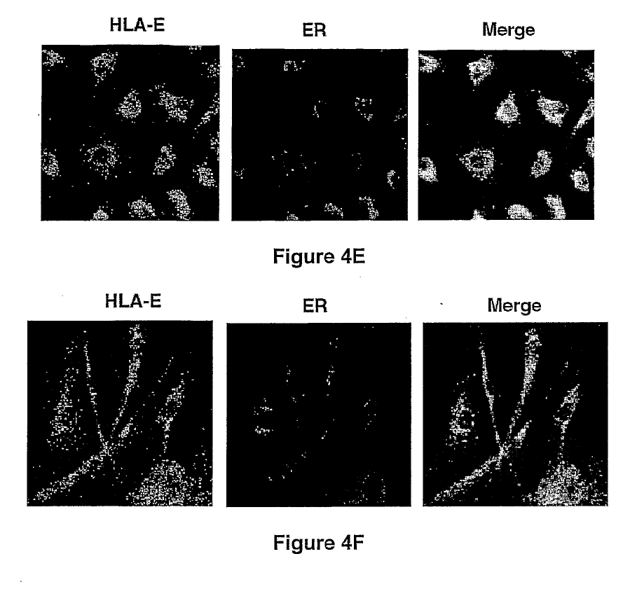

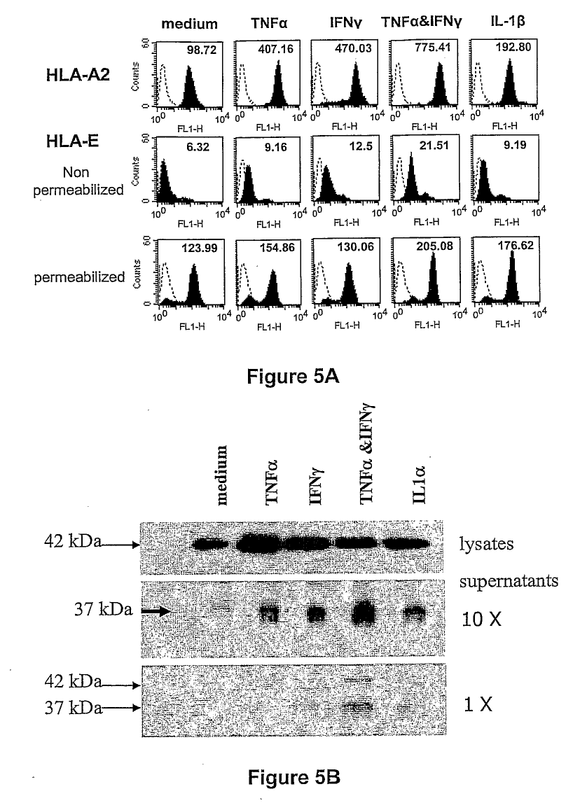

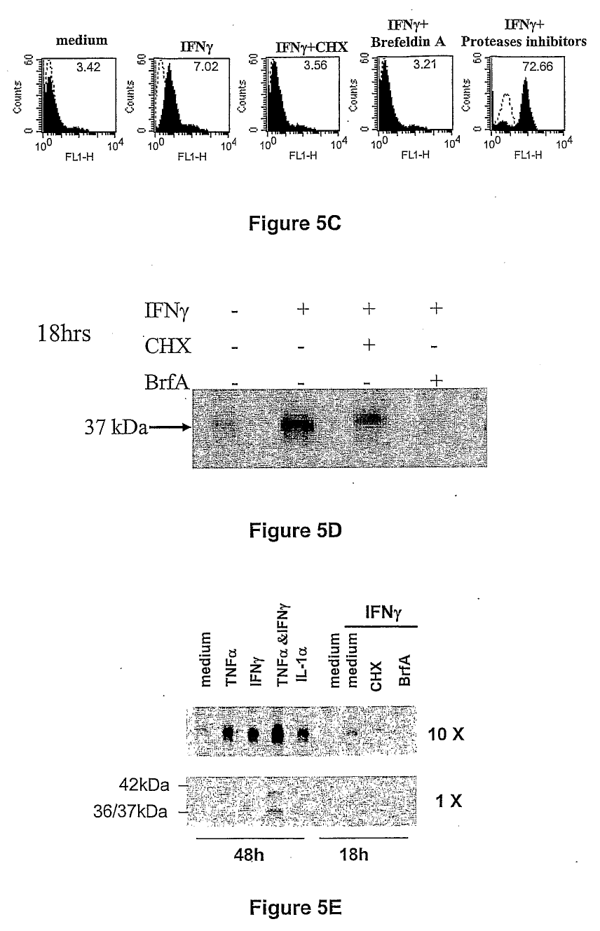

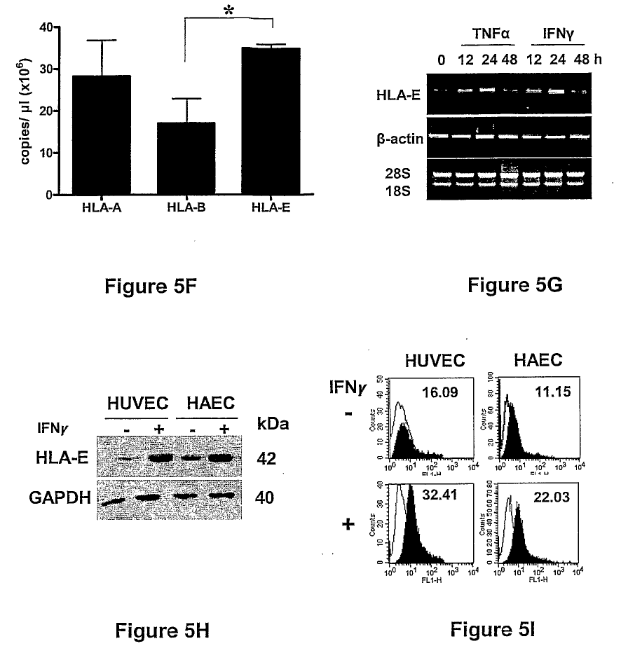

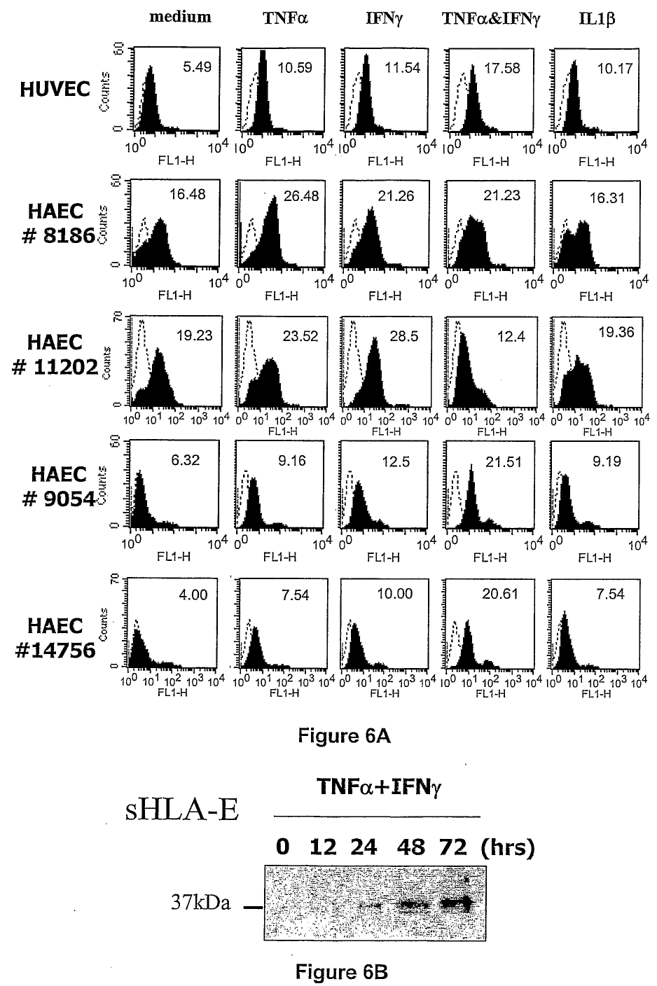

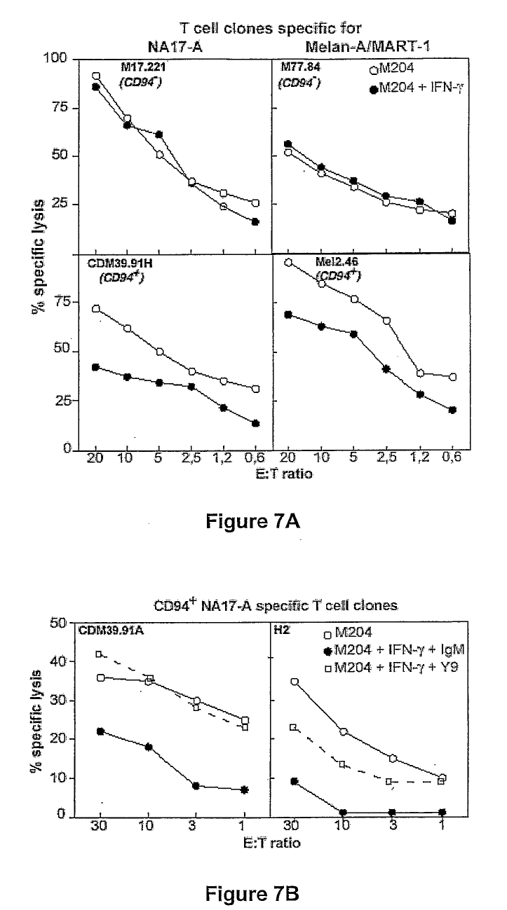

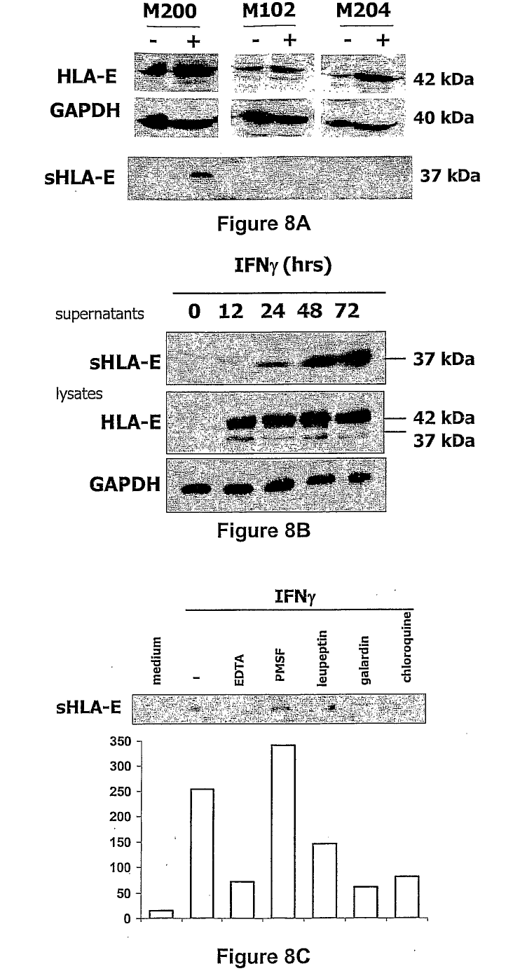

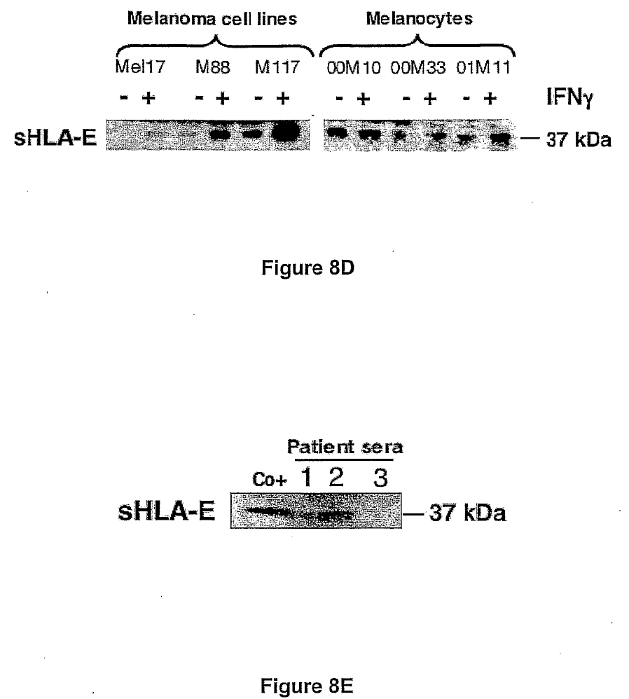

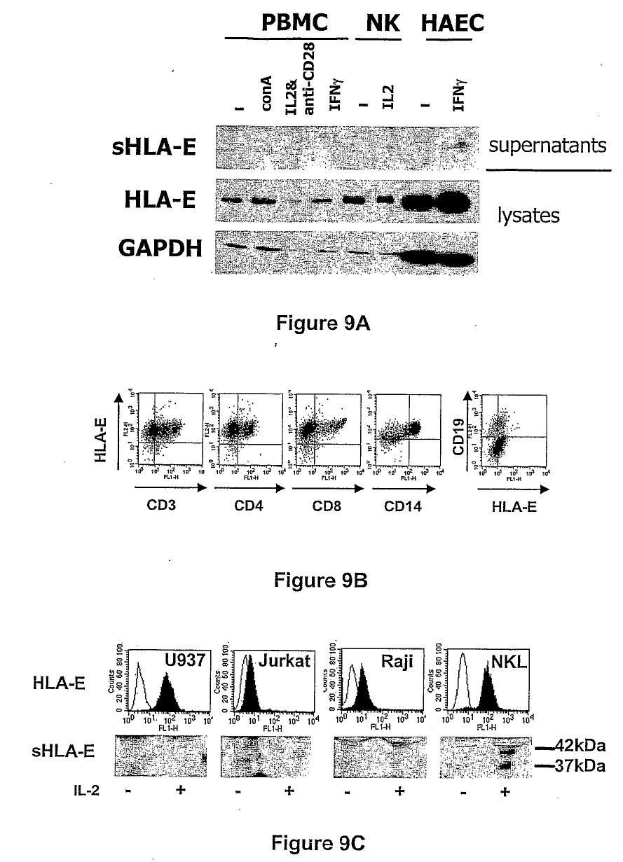

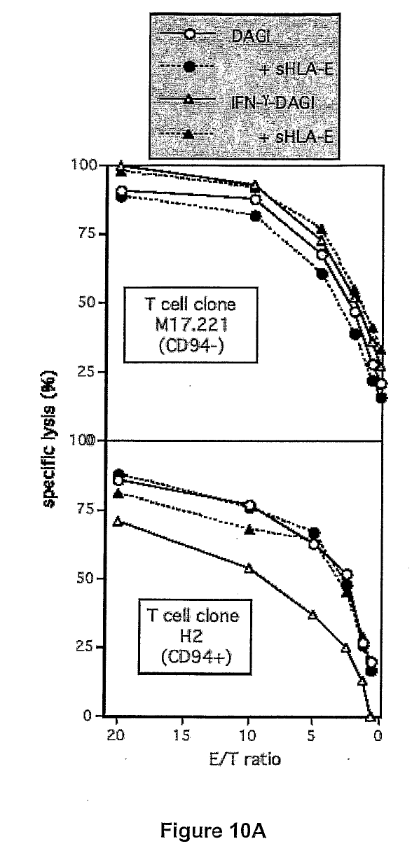

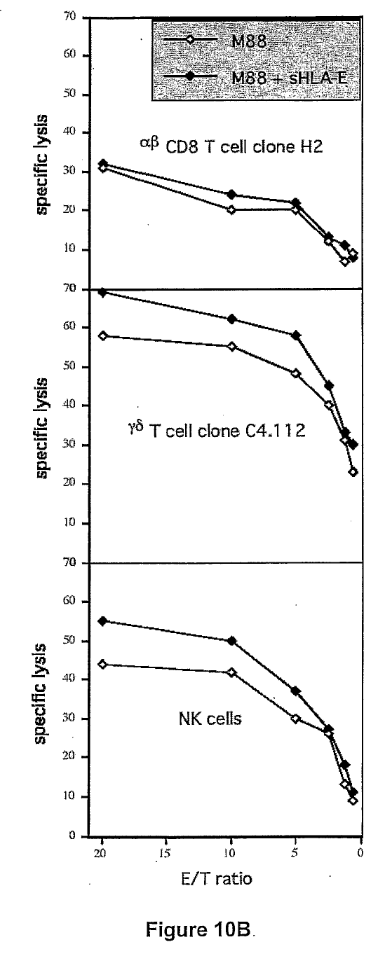

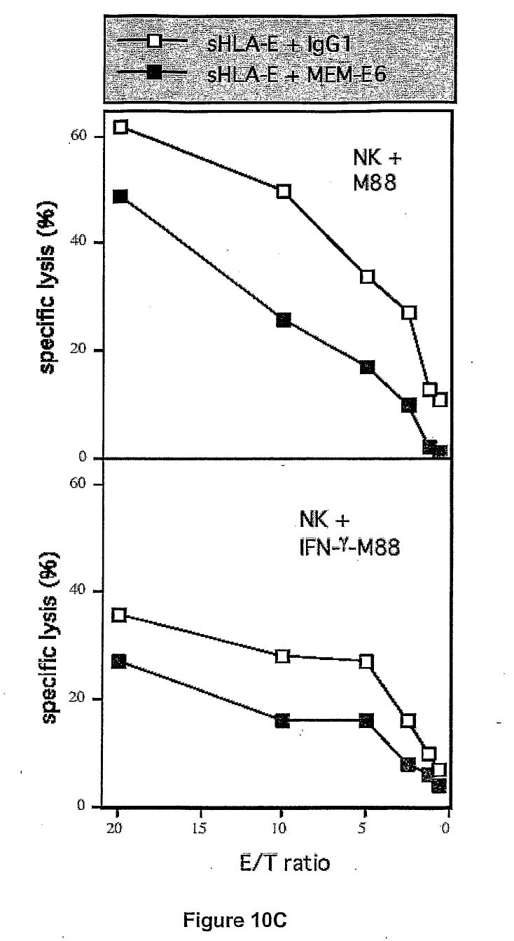

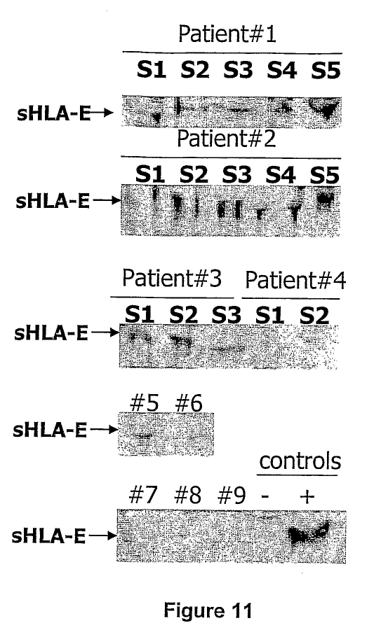

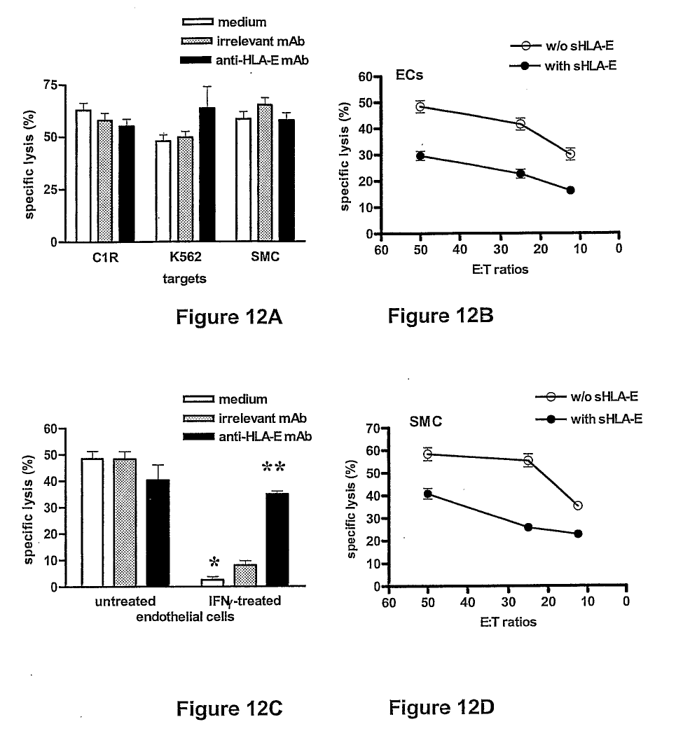

FIGS. 1A, 1B and 1C show the results obtained with the MEM-E/07 antibody specific for HLA-E in a panel of 22 melanoma cell lines. HLA-E expression was detectable although at low levels in all melanoma cell lines (as indicated by the ratio fluorescence intensity (RFI) ranging from 1 to 4). The hypothesis that this expression was due to the presence of HLA-B7 on melanoma cells, in view of cross-reaction described with the MEM-E7 Ab, was excluded.

It was observed that IFNγ up-regulates the HLA-E expression on the majority of melanoma cell lines (FIGS. 1A, 1B and 1C). However, no modulation of HLA-E expression was noted in approximately 20% of melanoma cell lines tested, independently of the up-regulation of total HLA Class I molecules by IFNγ.

All short term cultured melanocytes tested (FIGS. 1D and 1E) were clearly labeled by the anti-HLA-E mAb (as indicated by the RFI ranging from 3 to 7). However, this HLA-E labelling was upregulated only slightly by IFNγ treatment and only in half of them.

Therefore, all melanoma cell lines express low levels of HLA-E on their membrane and this expression is up regulated by IFNγ. In contrast, short cultured melanocytes significantly express HLA-E, but this expression is poorly increased by IFN-γ.

### Example 2

### HLA-E Expression in Human Tissues

In the present study, using anti-HLA-E specific (MEM-E/02, MEM-E/07, MEM-E/08) mAbs, the Inventors showed that HLA-E expression in human non lymphoid organs is mainly restricted to endothelial cells (EC).

First, HLA-E expression in human tissues was examined by immunohistochemistry using monoclonal antibodies specific for HLA-E (MEM-E/02) or HLA-G (MEM-G/01), as a control.

Comparative expression of HLA-E and HLA-G proteins were studied in human first-trimester placenta tissue and kidneys. HLA-E expression was also studied in various normal tissues included epithelial tissues (salivary gland, urinary bladder, thyroid, endometrium, skin, liver), kidney biopsies (obtained from patients suffering of lupus nephritis and vasculitis), lymphoid organs (lymph node and spleen), mesenchymal tissues and hematopoietic cells. Tissues were fixed in 10% formalin and routinely processed for paraffin embedding. Four micrometers-thick paraffin sections were then mounted on pre-treated slides, deparaffinized using toluene, rehydrated through a graded series of ethanol, and rinsed in distilled water. Tissue sections were then subjected to epitope retrieval in microwave oven using citrate buffer (pH 6.0). Tissue sections were stained using a two-step visualization system based on a peroxidase-conjugated dextran backbone, which avoid endogenous biotin detection (Dako Envision+™ System, Dako, Trappes, France). The following antibodies were used: mouse monoclonal anti-human HLA-E (MEM-E/02) and anti human HLA-G (MEM-G/1) mouse monoclonal Abs. Working dilutions were 1:100 for both. Tissue sections were rinsed in buffer, then endogenous peroxidase activity was blocked with the peroxidase-blocking solution for 5 min. Sections were then incubated with primary antibody at room temperature for 30 min. This was followed by incubation with secondary antibody coupled to the peroxidase-conjugated polymer for 30 min at room temperature. Immunostaining was visualized using the substrate system provided in the kit (DAB/H202 substrate) and tissues were counterstained with haematoxylin. Immunopositive cells showed cytoplasmic staining with both antibodies.

In placenta, comparative immunostaining indicated that HLA-E, as well as HLA-G (FIGS. 3A, 3G) was expressed in extravillous trophoblast whereas perivillous trophoblast and syncitiotrophoblast were negative. In addition, HLA-E, but not HLA-G, staining was also observed on endothelial cells. Endothelial expression for HLA-E was confirmed in kidney biopsies where HLA-E was consistently expressed in macrovascular, capillary and glomerular ECs (FIGS. 3B, 3C, 3D). No staining was observed on mesenchymal, tubular, mesangial cells, muscle cells or adipocytes. HLA-E staining was consistently observed on all EC from all types of vessels including arteries, veins, capillaries, and lymphatics. Endothelial expression for HLA-E was further observed in high endothelial venules in spleen and lymph node (FIGS. 3E, 3F) concomitant with a strong expression in B and T lymphocytes and in monocytes/macrophages (see also FIGS. 3I, 3J). Megakaryocytes but not erythrocytes also expressed HLA-E (see FIGS. 3I, 3J). HLA-E distribution among mesenchymal, epithelial, hematopoietic cells is summarized in Table 1.

TABLE 1
HLA-E expression in various normal tissues

| | HLA-E negative | HLA-E positive |
|---|---|---|
| Mesenchymal | Fat cells Fibroblasts Smooth | Endothelial cells of |
| cells | muscle cells (vascular wall, | arteries, veins, |
| | muscularis of urinary bladder | lymphatics Macrophages |
| | and stomach) Striated muscle | |
| | cells Peripheral nerves and | |
| | ganglion cells | |
| Epithelial | Salivary acinis and ducts | Few urothelial cells |
| cells | Thyroid follicular cells Liver | of urinary bladder, |
| | Epithelial skin appendages | strong staining of |
| | | secretory endometrial |
| | | cells during pregnancy. |
| Lymphoid | Interdigitated cells | Lymphoid B-cells |
| tissues | | (lymphoid follicle), |
| (lymph node | | lymphoid T-cells, |
| and spleen) | | macrophages, endothelial |
| | | cells of postcapillary |
| | | venules, sinus of |
| | | the spleen |
| Hematopoietic | Erythroblasts | Megakaryoblasts |
| cells | | |

To determine whether this staining observed in tissues indicated the presence of HLA-E on the extracellular side of the plasma membrane, immunofluorescence studies on non-permeabilized cultured ECs were performed.

For immunofluorescence, ECs were grown to confluence on glass coverslips. Cultures were washed with PBS and fixed for 20 min in PBS containing 4% paraformaldehyde. Cells were washed again with PBS and incubated over-night at 4° C. with blocking buffer (5% BSA in PBS) and then incubated with an anti-HLA-E mAb (MEM/E-7: 10 μg·ml⁻¹) in blocking buffer with 0.1% Triton X-100 (permeabilized) or without Triton X-100 (non-permeabilized) for 1 h. Slides were rewashed and incubated with FITC-conjugated goat anti-mouse antibodies (5 μg·ml⁻¹, Jackson Lab., West Grove, Pa.) for 1 h. ER and Golgi staining were preformed using rhodamine-B-hexyl ester (2.5 μg·ml⁻¹, Molecular Probes, Eugene, Oreg.) and anti-golgi mAbs (5 μg·ml⁻¹; anti-golgin-97, clone CDF4, Molecular Probes), respectively. Anti-golgi mAbs were revealed using TRITC-conjugated goat anti-mouse antibodies (5 μg·ml⁻¹, Jackson Lab., West Grove, Pa.). Nuclear staining was performed using To-Pro-3 (1:1000 dilution, Molecular Probes). Slides were washed in PBS, dried and mounted with ProLong®& antifade reagent (Molecular Probes). Fluorescence microscopy was performed with a Leica DM-IRBE® laser scanning confocal microscope (Leica AG, Heerbrugg, Switzerland) using a 63×1.4 oil p-Aplo lens and analyzed using Leica TCS NT® software.

The intracellular location of HLA-E was studied by immunofluorescence and confocal microscopy on cultured ECs. Although weaker in intensity than staining for HLA-A, -B and -C, HLA-E staining was found on non-permeabilized cells (FIG. 4B), implying that HLA-E is localized at the outer surface of the cells. It was also found that HLA-E has a perinuclear distribution in permeabilized ECs where HLA-E displays a co-localization with the endoplasmic reticulum and the Golgi (FIGS. 4C, 4D, 4E and 4F), suggesting that a form of HLA-E might be secreted.

### Example 3

### Cytokine-Induced Up-Regulation of HLA-E Cell-Surface Expression on Endothelial Cells

A quantification of HLA-A, -B and -E mRNA levels was performed in cultured ECs by competitive RT-PCR using locus-specific primers and a competitor template with an internal deletion. RNA was isolated using Trizol reagent (Invitrogen Corp.) according to the instructions of the manufacturer. RNA was analyzed by competitive PCR as described previously (Vincent et al (1996) J Immunol 156, 603-610). Briefly, total RNA (2 μg) was reverse transcribed with oligo (dT), treated with RNase H, and made up to 50 μl. cDNAs were diluted 1/2 for competitions. Competitor templates were initially diluted 1/106, followed by four serial dilutions of 1/3. The primer sequences were:

HLA-A (334 bp):

| | |
|---|---|
| sense: | 5′-CTACCCTGCGGAGATCA-3′, |
| antisense: | 5′-GCTCCCTCCTTTTCTATCTG-3′, |

HLA-B (255 bp):

| | |
|---|---|
| sense: | CTACCCTGCGGAGATCA, |
| antisense: | ACAGCCAGGCCAGCAACA, |

HLA-E (257 bp):

| | |
|---|---|
| sense: | 5′-CTACCCTGC GGAGATCA-3′, |
| antisense: | 5′-AGAGAACCAGGCCAGCAAT-3′, |

HPRT (78 bp):

| | |
|---|---|
| sense: | 5′-GGACAGGACTGAACGTCTTGC-3′, |
| antisense: | 5′-TTGAGCACACAGAGGGCTACA-3′. |

PCR products were sequenced by Genosys (Sigma). Internal standards were obtained by mutagene PCR amplications to generate mutated fragment by the deletion of 5 nucleotides as described (Vincent et al (1996) J Immunol 156, 603-610). PCR products were run on a 4% acrylamide gel and analyzed by capillary electrophoresis on an ABI PRISM 310 DNA Sequencer (PE Applied Biosystem, Foster City, Calif.) using GeneScan® Analysis software.

Semi-quantitative PCR for HLA-E and Pactin was carried out for 20 and 18 cycles, respectively, as follows: 94° C. for 30 seconds, 60° C. for 30 seconds, and 72° C. for 30 seconds, with a final extension at 72° C. for 3 minutes. PCR primers were:

HLA-E sense:
-5′-CCACCATGGTAGATGGAACCC-3′,
HLA-E antisense:
5′-GCTTTACAAGCTGTCAGACTC-3′,
β-actin sense:
5′-AATCTGGCACCACACCTTCTACA-3′,
β-actine antisense:
5′-CGACGTAGCACAGCTTCTCCTTA-3′.

PCR products were separated on a 1.5% agarose gel in the presence of ethidium bromide.

Transcripts encoded by all three HLA loci were detected in resting ECs, demonstrating that ECs express HLA-E constitutively. Whereas there are less HLA-B transcripts than HLA-A transcripts, ECs express HLA-E at levels nearly equal to those of HLA-A (FIG. 5F). This pattern of expression was observed in separate cultures of ECs issued from three individual donors. Both TNFα and IFNγ increased HLA-E transcripts with a maximal effect at 24 h (FIG. 5G).

Cell surface expression for HLA-E was further investigated by flow cytometry on cultured ECs (isolated from artery: HAEC and from umbilical vein: HUVEC) using MEM-E/7 or MEM-E/8 mAbs. Since inflammation deeply affects endothelial cell phenotype and functions, the effects of pro-inflammatory cytokines TNFα, IFNγ and IL1β on HLA-E expression and regulation were studied.

Briefly, human umbilical vein ECs (HUVEC) and arterial endothelial cells (HAEC), isolated from renal artery patches taken from cadaveric transplant donors before kidney transplantation, were isolated as previously described²⁸. ECs were HLA-typed and selected to avoid non-HLA-E-specific cross-reactivity with MEM/E-7 reported for HLA-B7. ECs were cultured in Endothelial Cell Growth Medium (ECGM) supplemented with 10% fetal calf serum (FCS), 0.004 ml·ml⁻¹ ECGS/Heparin, 0.1 ng·ml⁻¹ hEGF, 1 ng·ml⁻¹ hbFGF, 1 μg·ml⁻¹ hydrocortisone, 50 μg·ml⁻¹ gentamicin and 50 ng·ml⁻¹ amphotericin B (C-22010, PromoCell, Heidelberg, Germany). For activation, confluent EC monolayers were incubated with recombinant human TNFα(100 U·ml⁻¹), IFNγ(100 U·ml⁻¹), IL1β (5 ng·ml⁻¹) for the indicated period of time in Endothelial Cell Basal Medium supplemented with 2% FCS. For experiments, ECs were used between the second and fifth passage. Culture supernatants were collected at the indicated times post-activation and kept frozen. When needed, culture supernatants were concentrated (10×) using Microcon YM-3 (Millipore, Bedford, Mass.).

For flow cytometry, cells (1-2×10⁵ cells/sample) were suspended with Trypsin-EDTA (Gibco BRL), washed twice with PBS containing 1% BSA and 0.1% NaN₃, and then incubated on ice for 30 min with a saturating concentration of first antibody. After three washes in 1% BSA/0.1% NaN₃/PBS, cells were incubated with a FITC-labeled goat anti-mouse F(ab′)2 IgG (Jackson Lab., West Grove, Pa.) at 4° C. for 30 min. This step was followed by three washes in cold 1% BSA/0.1% NaN₃/PBS, and cells were suspended in 1% paraformaldehyde in PBS. Negative controls were performed by incubating the cells with isotype-matched control antibody. Mouse monoclonal antibodies (mAbs) used for this study were anti-pan HLA class I (clone W6/32) (ATCC), monomorphic anti-HLA-A2/A28 (clone), anti-HLA-E (MEM/E7 or MEM-E/08), PE or FITC-labeled anti-CD3, anti-CD4, anti-CD8, anti-CD19, anti-CD56, anti-CD14 (all purchased from BD Biosciences, Mountain View, Calif.). Fluorescence was measured on 10,000 cells/sample using a fluorescence activated cell sorter (FACScalibur®: Becton Dickinson, Mountain View, Calif.) and analyzed using CellQuestPro® software (Becton Dickinson). Data are depicted in histograms plotting mean fluorescence intensity (MFI) on a four-decade logarithmic scale (x-axis) versus cell number (y-axis).

Western blot analysis on cultured ECs revealed a single band at 42 kDa, consistent with the molecular weight for HLA-E protein and distinct from classical HLA class I protein (data not shown). Basal levels of cell surface HLA-E protein were low but significant and consistently observed on HAECs and HUVEC (FIGS. 5H and 51). In response to IFNγ, cell surface HLA-E levels were increased and reached a maximal level at 48 h after stimulation (FIGS. 5H and 51).

Treatment of ECs with TNFα, IL1β and IFNγ resulted in a significant increase in membrane-bound HLA-E detected by flow cytometry (FIG. 5A) and total HLA-E level (42 kDa) assessed by western blot analysis (FIG. 5B). Combined treatment with TNFα and IFNγ produced a further increase in HLA-E expression. Similar results were obtained on HUVEC and on vascular ECs isolated from 4 individuals (see FIG. 6A). HLA-E analysis after permeabilization of the cells (FIG. 5A) indicated that HLA-E was present at higher amounts inside the cells than on EC surface. However, regulation of intracellular HLA-E in response to cytokine was low.

### Example 4

### HLA-E Expression in Human Melanoma

In the present study, using anti-HLA-E specific (MEM-E/02, MEM-E/07, MEM-E/08) mAbs, the Inventors showed HLA-E expression in human melanoma in vitro and in vivo.

First, HLA-E expression in melanoma was examined by immunohistochemistry using monoclonal antibodies specific for HLA-E (MEM-E/02).

Comparative expression of HLA-E protein was studied on sections of 5 normal skin samples and of 11 melanoma tumor samples.

In all the normal skin sections, HLA-E was consistently expressed by melanocytes while a faint expression was observed in the epidermis with level variations according to samples and individuals (FIG. 2A and FIG. 2B). A strong expression for HLA-E was observed in cutaneous melanoma tumors where all transformed melanocytes expressed high levels of HLA-E (FIGS. 2C and 2D). The fraction of labeled tumor cells in these tumors ranged from 30% (in one tumor) to 70% (in three tumors). In metastasis, HLA-E expressing cells were scattered and accounted for 10 to 30% of the tumour cells in either invaded lymph nodes (FIGS. 2E and 2F), liver or gastric metastasis (FIGS. 2G and 2H). These data indicate that HLA-E expression vary during tumor progression. These data may also suggest that HLA-E expression could protect melanoma cells in primary tumors favoring tumor cells migration and organ invasion.

### Example 5

### HLA-E-Mediated Protection from CTL-Lysis of IFNγ-Treated Melanoma Cell Lines

The Inventors then questioned whether HLA-E density on melanoma cells could modulate the susceptibility of these cells to lysis by melanoma Ag specific CTL clones expressing the CD94/NKG2-A receptor. To this end, the lysis of different melanoma cell lines treated or not by IFNγ by melanoma specific CTL clones, expressing or not the inhibitory receptor, was compared.

Briefly, melanoma-reactive CD8 αβ T cell clones specific for Melan-A/MART-1 (M77.84, M199.7.5, M17.29ELA.1, M17.29ELA.3, MEL.C8, and MEL2.46), tyrosinase (M117.14) and NA17-A (M17.221, D126, H2, MEL.F5, CDM39.91A and CDM39.91H) antigens were obtained from TIL (Tumor Infiltrating Lymphocytes) or from PBL (Peripheral Blood Lymphocytes) stimulated ex vivo by peptide pulsed antigen presenting cells (APC). The γδ T cell clone C4.112 was obtained from colon tumor-infiltrating lymphocytes. T cell clones were expanded using a polyclonal T cell stimulation protocol, as described previously (Jotereau et al. (1991) J Immunother 10, 405-11). Briefly, 2000 T cell clones were distributed/well in 96 wells-plates with 200 μl of culture medium (RPMI with 8% human serum and IL-2 150 U/ml) and irradiated feeder cells: LAZ EBV-B cells (2·10⁴/well), allogeneic PBL (10⁵/well) and 15 μg/ml of PHA-L (Difco, Detroit, USA). Peripheral blood polyclonal NK cells were sorted by negative isolation kit from Miltenyi.

Representative examples are shown on FIG. 7A. Upon IFNγ treatment, lysis of M204 cell line by CD94/NKG2A-expressing melanoma Ag (NA17-A or Melan-A/MART-1) specific CTL clones was significantly decreased, while lysis by the clones devoid of CD94/NKG2-A remained unaffected. This protection of IFNγ-treated cells was mediated by the interaction of HLA-E and CD94/NKG2A, because a blocking anti-CD94 mAb (Y9) totally or partially reconstituted lysis (FIG. 7B). Transfection of melanoma cell lines by HLA-E cDNA significantly decreased the specific production of TNF by CD94/NKG2A⁺ T cell clones confirming the implication of the CD94/NKG2A/HLA-E-mediated inhibition. It was also observed that the level of melanoma cell lysis inhibition by antigen specific CTL induced by IFNγ treatment depends both on the density of the CD94/NKG2-A receptor and on the TCR dependent lytic potential of these CTL.

### Example 6

### Soluble HLA-E (sHLA-E) Secretion by Melanoma Cells and Melanocytes

It was then investigated if soluble HLA-E (sHLA-E) proteins might be secreted by melanoma cells and melanocytes, either spontaneously or after INFγ treatment. HLA-E proteins were studied by Western blot analysis in supernatants of a panel of melanoma cell lines cultured 2 days in the presence or not of IFNγ (500 U/ml), using the MEM-E/02 mAb that reacts specifically with the denaturated heavy chain of human HLA-E.

For Western blot analysis, aliquots of total cells supernatants, lysates or sera were separated in 12% SDS-PAGE. The gels were blotted onto nitrocellulose membranes (Hybond; Amersham, Buckinghamshire, U.K.), and the membranes were blocked by incubation with TBS containing 0.1% Tween 20 and 5% nonfat dry milk. The membranes were then probed with the anti-HLA-E Ab (MEM-E2) overnight at 4° C. and washed in TBS containing 0.1% Tween 20. After washing, the membranes were incubated for 2 hours at room temperature with peroxidase-conjugated sheep anti-mouse IgG Ab (Ozyme), washed thoroughly during 2 hours, stained with enhanced chemiluminescence reagent (Roche), and exposed to x-ray film. Cells were stimulated by IFNγ (500 U/ml) for various times. In some experiments, cells were stimulated by IFNγ for 24 h and cultured for a further 4 h with chloroquine (100 μM), leupeptin (100 μM), PMSF (1 mM), EDTA (0.5 mM), Galardin (1 mg/ml).

The results revealed a band of 37 kDa, corresponding to the detection of a soluble HLA-E form (see for examples FIG. 8A), in 13 of 22 melanoma cell lines tested upon IFNγ treatment. No or low level of soluble HLA-E was observed in supernatants of untreated melanoma cells supernatant. In contrast, soluble HLA-E was systematically detected in supernatants of untreated melanocytes (FIG. 8D). Concomitantly, HLA-E proteins were studied by Western blot analysis in all lysates confirming the HLA-E expression by all the melanoma cell lines, and their augmentation after IFNγ treatment. Kinetic analysis showed that a maximum level of sHLA-E was found 48 hours after the beginning of treatment with IFNγ (FIG. 8B). The Inventors next investigated whether the release of soluble HLA-E by melanoma cells involves a proteolytic cleavage. The IFNγ-pretreated M200 cell line was cultured for 4 h with chloroquine (lysosomal inhibitor), leupeptin (Ser/Thr proteinase inhibitor), PMSF (thiol proteinase inhibitor), EDTA (metalloproteinase inhibitor) or Galardin (MMP inhibitor). The level of soluble HLA-E in the culture supernatant was quantified. Treatment of cells with leupeptin or PMSF did not affect the generation of soluble HLA-E, whereas, treatment of cells with EDTA, chloroquine or Galardin markedly reduced the release of soluble HLA-E (FIG. 8C). These results indicate that the soluble form of HLA-E is generated by metalloproteinase-dependent shedding.

### Example 7

### Soluble HLA-E (sHLA-E) Secretion by Endothelial Cells

Western blotting also revealed the presence of soluble HLA-E (sHLA-E) in the supernatants of activated ECs.

Briefly, cells were maintained for 12-18 h in culture medium without growth supplements and containing 2% serum before treatment. When applicable, cells were treated for 12-72 h with 100 U·ml⁻¹ TNFα, 100 U·ml⁻¹ IFNγ, and/or 2.5 ng·ml⁻¹ IL1β. When applicable, cells were incubated with brefeldin A, galardin or protease inhibitors (Sigma-Aldrich, Saint Quentin Fallavier, France) for the last 6 h. Alternatively, cells were treated for 2 h with cyclohexamide, actinomycin D, monensin or tunicamycin before stimulation. Supernatants were collected and centrifuged for 10 min at 1,500 g to remove cell debris and then used immediately or stored at −70° C. Cells (3×10⁶) were washed in PBS and incubated in 300 μl lysis buffer (20 mM Tris-HCl, pH 7.4, 137 mM NaCl, 0.05% Triton X-100 and complete protease inhibitor cocktail (Sigma-Aldrich)) for 15 min on ice. Cell lysates were centrifuged at 12,000 g for 10 min at 4° C. the supernatants were then recovered and the protein extracts were used immediately or stored at −70° C. Equal amounts of protein (15 μg) were loaded under reducing conditions and resolved by 12% SDS-PAGE gels. Western immunoblot analysis were performed on nitrocellulose membranes (Amersham-Pharmacia, Orsay, France) using anti-HLA-E monoclonal antibodies (MEM/E-2). Anti-mouse IgG, HRP-linked Abs (Cell Signaling Technology, Beverly, Mass.) were used as secondary Abs in cheluminescent western blot assays using ECL® system (Amersham-Pharmacia). When applicable, blots were reprobed with mouse monoclonal anti-GAPDH Ab (Chemicon, Temecula, Calif.) to verify the amount of loaded proteins.

HLA-E release is driven by TNFα or IL1β and the combination of IFNγ and TNFα have an additive effect on its surface expression and release (FIG. 5B). Soluble HLA-E was detected as a major band of 37 kDa (a doublet of 36 and 37 kDa) corresponding to metalloproteinase-dependent shedding of membrane bound protein but also as a band of 42 kDa corresponding to full length protein which probably results from alternative splicing as reported for other soluble HLA molecules (Demaria et al. (2000) Hum Immunol 61, 1332-8; Haynes et al. (2002) Hum Immunol 63, 893-901). Soluble HLA-E protein was detectable after 12-24 h of activation and maximal at 72 h (FIG. 6B). Both actimycin D and cyclohexamide prevented HLA-E surexpression and release by activated ECs suggesting that soluble HLA-E requires mRNA and protein synthesis (FIGS. 5C, 5D and 5E). Presence of brefeldin A, a specific inhibitor of exocytosis, also inhibited both the increase of cell surface expression and the release of HLA-E. In contrast, protease inhibitors abrogated HLA-E secretion, while significantly increased expression of HLA-E at the membrane confirming that EC, at least in part, generate sHLA-E by proteolytic shedding. Therefore, EC activation by inflammatory cytokines concurs to both increased membrane-bound HLA-E expression and release of sHLA-E.

### Example 8

### Cellular Expression and Release of Soluble HLA-E (sHLA-E) Expression by Cells of the Immune System

HLA-E expression by leucocytes was confirmed in vitro by flow cytometry performed on fresh PBMC. FIG. 9B shows that HLA-E was consistently expressed by CD3+, CD4+, CD8+, CD14+ and CD19+ leucocyte subsets.

It was next examined whether they could also produce soluble HLA-E. To this aim, culture supernatants were collected from either non-stimulated or 48 h-activated fresh human PBMC (Peripheral Blood Mononuclear Cells) or purified subsets and subjected to Western blotting.

Briefly, PBMC from random healthy volunteers were purified by Ficoll/Hypaque density centrifugation. NK cells were purified (>95% of CD3-CD56+ and/or CD16+) by negative selection using the NK Cell Isolation kit according to manufacturer's recommendations (Miltenyi Biotec, Paris, France). The human NK cell line, NKL, was grown in RPMI 1640 media (Gibco BRL, Life technologies, Cergy-Pontoise, France) supplemented with 10% FCS, 4 mM glutamine, 1 mM sodium pyruvate, and 200 U/ml of rIL-2 (R&D Systems, Lille, France). U937, Raji, and Jurkat cell lines were from American Tissue Culture Collection (Rockville, Md.). NKL cell line was kindly provided by Dr. Eric Vivier (CIML, Marseille-Luminy, France).

The presence of the soluble HLA-E protein was not detected in most conditions excepted for purified NK cells activated with IL-2 (FIG. 9A). Similarly, soluble HLA-E was not found in culture supernatants from resting or IFNγ-treated monocyte (U937), B (Raji) and T (Jurkat ) cell lines, but was present in significant amount in the culture supernatant from a NK cell line (NKL) (FIG. 9C). These data indicate that among HLA-E expressing cells, the production of sHLA-E is limited to a restricted set of cells including endothelial and NK cells.

### Example 9

### Purification of Soluble HLA-E Molecules (SHLA-E) Form Cell Culture Supernatants

Soluble HLA-E (sHLA-E) was purified from culture supernatants according to the following procedure.

Solid ammonium sulfate was be added to the culture supernatant at 4° C. to 40% saturation, the resulting solution was centrifuged and the pellet discarded, additional ammonium sulfate was then added to resulting supernatant to achieve 70% saturation. After centrifugation, the soft pellet was be placed in dialysis bags and dialyzed overnight against 12 volumes of 20 mM Tris-HCl, 0.02% sodium azide, pH 7.4. The dialysate was applied to an anti-HLA-E mAb AffiGel-10 immunoaffinity column (6 ml resin; 5 mg IgG/ml resin) equilibrated in 20 mM Tris-HCl, 0.1 M NaCl, 0.02% sodium azide, pH 7.4. The column was washed with at least 12 volumes of the same buffer, and then eluted with 50% (vol/vol) ethylene glycol in 20 mM Tris-HCl, pH 7.4. The peak fractions from the elution were pooled, concentrated (Centriprep 30; Amicon Inc., Beverly, Mass.), and the buffer changed to 20 mM Tris-HCl, 0.1 M NaCl, 3 mM CaCl₂, 0.6 mM MgCl₂, 0.02% sodium azide, pH 7.4. Fractions were monitored for absorbance at 280 nm, and for sHLA-E antigen using an ELISA assay. Purity of sHLA-E was assessed on SDS-PAGE gels with Coomassie BB staining, and on Western blots with anti-sHLA-E specific antibody.

### Example 10

### sHLA-E Induced Melanoma Lysis

To assess the functional role of sHLA-E, melanoma-specific T cell clones were pre-incubated with sHLA-E-positive melanoma supernatant before they were tested against IFNγ-treated melanoma cell target.

Standard 4-h ¹⁵Cr-release assays were used to assess antigen-specific target cell lysis. Target cells (human melanoma cell lines or B-EBV cell lines) were labeled with 100 μCi Na₂⁵¹CrO₄ (Oris Industrie, Gif-sur-Yvette, France) for 1 h at 37° C. 1000 or 5000 labeled target cells were incubated in the presence of T cell clones at different lymphocyte:target cell ratios (from 0.6 to 20). For some experiments, the melanoma target cells were previously stimulated with IFNγ (500 U/ml) for 48 h to increase MHC expression. In blocking experiments, the effector cells were preincubated with Y9 Ab (pure hybridoma supernatant) or irrelevant isotype control (1 μg/ml), sHLA-E containing supernatants or irrelevant supernatant (pure) at 4° C. for 20 min. prior to addition to the labeled target cells.

Analysis by cytotoxicity assays showed that IFNγ-protection of DAGI from CD94⁺ T cell clones was suppressed by pre-incubation of T cells with sHLA-E-positive supernatant but not with sHLA-E-negative supernatant (FIG. 10A). In contrast, IFNγ-treated DAGI was killed by CD94⁻ T cell clones at similar levels than the untreated DAGI, independently of the addition of sHLAE-positive supernatant. To determine the importance of soluble HLA-E in immune responses, their effect on various tumor effector functions was tested. M88 melanoma cell line was chosen as target for its natural high expression of HLA-E molecules, shown by FACS analysis. It was observed that sHLA-E-positive supernatant increase the lytic responses of freshly isolated NK cells and a γδ T cell clone and more slightly of a CD8 αβ T cell clone (FIG. 10B).

The increase of cytolytic responses may be caused by the presence of soluble HLA-E in supernatants of IFNγ-treated melanoma cells. This hypothesis was confirmed by using the blocking anti-HLA-E mAb MEM-E6 which abrogated the effect of sHLA-E-positive supernatant on M88 (as well as IFNγ-treated M88) lysis by NK cells (FIG. 10C).

Thus, these data indicate that soluble HLA-E are physiologically active in modulating the responsiveness of different tumor effectors by increasing CD94/NKG2A⁺ cell activation.

### Example 11

### sHLA-E Presence in the Sera of Patients Afflicted with Vasculitis

To investigate the role of shedding of HLA-E as a potential immune control mechanism of inflammatory processes in vivo, sHLA-E levels in sera of patients with ANCA-associated vasculitis were analyzed (FIG. 11). Serum samples of healthy volunteers (n=10) and patients (n=13, 22 sera) were collected and assayed for sHLA-E.

Serum samples (n=22) from 13 patients with ANCA-associated vasculitis (Wegener's granulomatosis) or other autoimmune diseases (systemic lupus erythematosus) were analyzed. All patients with kidney involvement had renal biopsy-proven vasculitis. Serum samples were obtained in the active phase (at diagnosis or relapse) and in remission (3 to 9 months later). The study was performed according to the guidelines of the local ethics committee (CHU de Nantes, France). Sera from healthy blood donors were provided by EFS (Nantes, France) and used as controls.

The presence of sHLA-E in patient sera, examined by immunoblot analysis of sera, revealed a band comparable to that of sHLA-E in culture supernatants (37 kDa). Presence sHLA-E was not detected in all investigated sera of healthy volunteers. Sera from 9 out of 13 patients with vasculitis showed presence of sHLA-E. This strong correlation clearly suggests that HLA-E is released at significant amounts from cells in vivo.

Further results (Table 2 and Table 3) indicated that presence of sHLA-E in patient's sera was positively correlated with disease activity in anti-neutrophil cytoplasmic antibody (ANCA)-associated systemic vasculitis (Wegener's granulomatosis and microscopic polyangitiis).

TABLE 2
Correlation of soluble HLA-E in patient's sera and disease
activity in ANCA- associated systemic vasculitis (WG & MPA)

| | Sera | Soluble | | | Soluble |
|---|---|---|---|---|---|
| | (number | HLA-E | C-reactive | | MICA |
| | of | (number of | protein | sIL2-R | (number of |
| | patients) | positive sera) | (mg · l⁻¹) | (μg · ml⁻¹) | positive sera) |
| Total vasculitis | 23 (10) | | | | 0 |
| (Wegener & MPA) | | | | | |
| Active phase | 10 | 9* | 143 ± 69 | 5.6 ± 3.2** | 0 |
| Inactive phase | 13 | 3 | 3.4 ± 0.4 | 2.1 ± 0.9** | 0 |
| SLE | 2 (1) | 0 | | | |
| Active phase | 1 | 0 | ≦3.2 | 0.9 ± 0.5 | 0 |
| Inactive phase | 1 | 0 | ND | ND | ND |
| Sepsis | 3 (3) | 0 | 100 ± 14 | 14.0 ± 3.9** | 0 |
| Controls | 9 (9) | 0 | ≦3.2 | 0.65 ± 0.36 | 0 |

MPA: microscopic polyangiitis; SLE: systemic lupus erythematous
Soluble HLA-E was detected by Western blot assays; *P < 0.01 versus inactive phase
Soluble MICA was detected by Elisa (Immatics, Tübingen, Germany), threshold of detection: 30 pg ml⁻¹
Sera from patients with vasculitis (n = 23), sepsis (n = 3) or healthy controls (n = 9) were assayed for the IL-2 soluble receptor α by ELISA and the means were compared by Student's t-test with receptor levels found in normal sera. In both patient groups, we found significant elevations in soluble IL-2Rα levels relative to normal volunteers (**p < 0.01).

TABLE 3
Correlation of soluble HLA-E in patient's sera and disease
activity in ANCA-associated systemic vasculitis (WG & MPA)

| | | | C-reactive | | | |
|---|---|---|---|---|---|---|
| | Sera | Soluble | protein | sIL-2Rα | IL-8 | VEGF |
| | (patients) | HLA-E | (mg/l) | (μg/ml) | (pg/ml) | (pg/ml) |
| Total vasculitis | 22 (10) | | | | | |
| (WG & MPA) | | | | | | |
| Active phase | 11 | 9* | 143 ± 69*,** | 5.6 ± 3.2*,** | 585 ± 73* | 1055 ± 265*,** |
| Inactive phase | 11 | 3 | 3.4 ± 0.4 | 2.1 ± 0.9** | ≦30 | 214 ± 104** |
| Controls | 9 (9) | 0 | ≦3.2 | 0.65 ± 0.36 | ≦30 | ≦30 |

WG: Wegener's Granulomatosis, MPA: microscopic polyangiitis;
Soluble HLA-E was detected by Western blot assays;
*P < 0.01 versus inactive phase,
**P < 0.01 versus controls (sera from healthy donors)

Sera from patients with vasculitis (n=22) or healthy controls (n=9) were assayed for the IL-2 soluble receptor a, IL-8 and VEGF by ELISA and the means were compared by Student's t-test with receptor levels found in normal sera. In both patient groups, we found significant elevations in soluble IL-2Rα and VEGF levels relative to normal volunteers (**p<0.01).

### Example 12

### sHLA-E Presence in the Sera of Patients Afflicted with Melanoma

In the frame of the present invention, the presence of tumor-associated HLA-E was looked for in the circulation of healthy donors and melanoma patients. By a Western blot analysis, the presence of soluble HLA-E was detected in 18 of 29 blood serum samples obtained from patients bearing primary (n=14) or metastatic (n=15) melanoma tumors (see for examples FIG. 8E) while all 10 serum samples from healthy individuals gave negative results. Therefore, soluble HLA-E is specifically present in sera of melanoma patients.

### Example 13

### HLA-E-Mediated Protection from NK Cell-Mediated Cytotoxicity

The respective functions of membranous and soluble HLA-E molecules, expressed and released by ECs, were evaluated in cell-mediated cytotoxicity assays. To this aim, NK cells were purified from PBMC and used as effector cells in cytotoxicity assays where target cells were the class I-deficient cell lines C1R and K562, primary cultures of smooth muscle cells (SMC), which do not expressed HLA-E, or ECs (resting or activated for 48 h with IFNγ). Experiments were performed in the presence or absence of a blocking anti-HLA-E mAb.

As depicted on FIG. 12A, cells which express no HLA-E at the cell surface (C1R, K562 and SMC) were efficiently lysed by NK cells, with no effect of anti-HLA-E blocking mAb. Resting ECs were also efficiently killed by allogeneic NK and blocking HLA-E did not affect lysis of cells (FIG. 12C). Thus, basal level of surface HLA-E on non-activated ECs seems not sufficient to allow protection. EC activation with IFNγ dramatically decreased NK-mediated cytotoxicity as compared to resting ECs (2.6±0.5% versus 48.4±5.4% for IFNγ-treated and untreated ECs, respectively,*p<0.01). Blocking HLA-E on IFNγ-activated HAEC significantly restore cell lysis (34.9±3.4% versus 8.3±2.2% in the presence of anti-HLA-E and irrelevant Abs, respectively, **p<0.01), suggesting that up-regulation of HLA-E, at cell surface, upon IFNγ provides a protection against NK cytotoxicity.

In an attempt to determine the biological activity of sHLA-E, cytotoxic activity of NK cells toward cells with no or low HLA-E expression (untreated EC and SMC) was measured in the presence of conditioned medium containing sHLA-E. These experiments indicated that cell lysis was significantly decreased when sHLA-E was present (FIGS. 12B and 12D). Altogether, these data suggest that up-regulation of HLA-E at the membrane of ECs confers protection against NK-mediated lysis, while production of sHLA-E may also protect cells with no or low expression of HLA-E, such as SMC or quiescent ECs.

1. A purified soluble HLA-E molecule, characterized in that:
it is a monomelic protein,
it presents a sequence identity of at least 80% with membrane-bound HLA-E (SEQ ID NO: 1),
it has a molecular weight from about 36 kDa to about 42 kDa,
it binds to the CD94/NKG2A receptor.

2. A process for obtaining a soluble HLA-E molecule, comprising a step of recovering soluble HLA-E molecules from a culture medium in which tumor cells, melanocytes, Natural Killer cells, and/or endothelial cells have been grown.

3. A process for obtaining a soluble HLA-E molecule according to claim 2, wherein the culture medium contains at least one cytokine.

4. A process for obtaining a soluble HLA-E molecule according to claim 3, wherein the cytokine is selected from the list comprising IFNγ, IL1β and TNFα.

5. A soluble HLA-E molecule such as obtainable according to the process of claim 2.

6. Ligands of soluble HLA-E molecules according to claim 1 which neither bind to membrane bound HLA-E molecules nor to bacterially produced soluble HLA-E molecules.

7. Ligands according to claim 6, characterized in that they are selected from the list comprising antibodies or paratope-containing fragments thereof, and aptamers.

8. An in vitro method for diagnosing cancers or inflammatory diseases in a patient, characterized in that HLA-E presence is detected in a biological sample, in particular a sample of solid tissues, such as skin, or liquid tissues, such as serum or plasma, taken from the patient.

9. An in vitro method for diagnosing cancers or inflammatory diseases in a patient according to claim 8, characterized in that the presence of soluble HLA-E is detected in a biological sample, in particular a sample selected from a sample of blood, serum, or plasma, taken from the patient

10. An in vitro method for diagnosing cancers or inflammatory diseases in a patient according to claim 8, characterized in that the presence of HLA-E is detected by contacting the sample taken from the patient with a HLA-E ligand.

11. An in vitro method for diagnosing cancers or inflammatory diseases in a patient according to claim 10, characterized in that the HLA-E ligand is selected from a list comprising anti-HLA-E antibodies or paratope-containing fragments thereof, anti-HLA-E aptamers, and CD94/NKG2A molecules or binding site-containing fragments thereof.

12. An in vitro method for diagnosing cancers or inflammatory diseases in a patient according to claim 8, characterized in that the cancers are melanomas, and the inflammatory diseases are vasculitides, in particular anti-neutrophil cytoplasmic antibody (ANCA)-associated systemic vasculitides.

13. A kit for diagnosing cancers or inflammatory diseases in a patient, characterized in that it comprises:
at least one HLA-E ligand,
a mean for detecting the binding of the HLA-E ligand to a soluble HLA-E,
optionally a soluble HLA-E, in particular a soluble HLA-E according to claim 1, as a standard.

14. A kit for diagnosing cancers or inflammatory diseases in a patient according to claim 13, characterized in that the HLA-E ligand is selected from a list comprising anti-HLA-E antibodies or paratope-containing fragments thereof, anti-HLA-E aptamers, and CD94/NKG2A molecules or binding site-containing fragments thereof.

15. A pharmaceutical composition, characterized in that it comprises at least one soluble HLA-E molecule according to claim 1 in association with a pharmaceutically acceptable vehicle.

16. The use of at least one soluble HLA-E molecule according to claim 1 for the manufacture of a medicament intended for the treatment of cancers, in particular melanomas.

17. A pharmaceutical composition, characterized in that it comprises at least one HLA-E ligand in association with a pharmaceutically acceptable vehicle.

18. A pharmaceutical composition according to claim 17, characterized in that the HLA-E ligand is selected from a list comprising anti-HLA-E antibodies or paratope-containing fragments thereof, anti-HLA-E aptamers, and CD94/NKG2A molecules or binding site-containing fragments thereof.

19. The use of at least one HLA-E ligand for the manufacture of a medicament intended for the treatment of inflammatory diseases, in particular vasculitides, such as anti-neutrophil cytoplasmic antibody (ANCA)-associated systemic vasculitides.

20. The use according to claim 19, characterized in that the HLA-E ligand is selected from a list comprising anti-HLA-E antibodies or paratope-containing fragments thereof, anti-HLA-E aptamers, and CD94/NKG2A molecules or binding site-containing fragments thereof.

21. The use of cytokines, in particular selected from the list comprising IFNγ, IL1/β and TNFα, for producing a soluble HLA-E molecule from cells in vitro.


SEQUENCE LISTING (14 sequences)

SEQ ID NO: 1 - protein, 364 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGHFLPTGGKGGSYSKAEWSDSAQGSESHSL

SEQ ID NO: 2 - protein, 179 aa, Homo sapiens
MAVFKTTLWRLISGTLGIICLSLMATLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 3 - protein, 233 aa, Homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 4 - DNA, 17 bp, Artificial Sequence
  Description of Artificial Sequence Synthetic primer
ctaccctgcggagatca

SEQ ID NO: 5 - DNA, 20 bp, Artificial Sequence
  Description of Artificial Sequence Synthetic primer
gctccctccttttctatctg

SEQ ID NO: 6 - DNA, 18 bp, Artificial Sequence
  Description of Artificial Sequence Synthetic primer
acagccaggccagcaaca

SEQ ID NO: 7 - DNA, 17 bp, Artificial Sequence
  Description of Artificial Sequence Synthetic primer
ctaccctgcggagatca

SEQ ID NO: 8 - DNA, 19 bp, Artificial Sequence
  Description of Artificial Sequence Synthetic primer
agagaaccaggccagcaat

SEQ ID NO: 9 - DNA, 21 bp, Artificial Sequence
  Description of Artificial Sequence Synthetic primer
ggacaggactgaacgtcttgc

SEQ ID NO: 10 - DNA, 21 bp, Artificial Sequence
  Description of Artificial Sequence Synthetic primer
ttgagcacacagagggctaca

SEQ ID NO: 11 - DNA, 21 bp, Artificial Sequence
  Description of Artificial Sequence Synthetic primer
ccaccatggtagatggaaccc

SEQ ID NO: 12 - DNA, 21 bp, Artificial Sequence
  Description of Artificial Sequence Synthetic primer
gctttacaagctgtcagactc

SEQ ID NO: 13 - DNA, 23 bp, Artificial Sequence
  Description of Artificial Sequence Synthetic primer
aatctggcaccacaccttctaca

SEQ ID NO: 14 - DNA, 23 bp, Artificial Sequence
  Description of Artificial Sequence Synthetic primer
cgacgtagcacagcttctcctta